(19) United States
(12) Patent Application Publication
(10) Pub. No.: US 2017/0105934 A1
(43) Pub. Date: Apr. 20, 2017

(54) AQUEOUS OPHTHALMIC COMPOSITION

(71) Applicant: ROHTO Pharmaceutical Co., Ltd., Osaka (JP)

(72) Inventors: Yoko Mizutare, Osaka (JP); Yasuko Matsumura, Osaka (JP)

(73) Assignee: ROHTO Pharmaceutical Co., Ltd., Osaka (JP)

(21) Appl. No.: 15/317,851

(22) PCT Filed: Jun. 9, 2015
(86) PCT No.: PCT/JP2015/066606

§ 371 Date: Dec. 9, 2016

(30) Foreign Application Priority Data
Jun. 10, 2014 (JP) 2014-119885
Apr. 15, 2015 (JP) 2015-083703

(51) Int. Cl. A61K 9/08; A61K 47/12; A61K 47/10; A61K 47/18; A61K 47/02; A61K 9/00; A61K 47/26

(52) U.S. Cl. CPC A61K 9/08; A61K 9/0048; A61K 47/12; A61K 47/26; A61K 47/18; A61K 47/02; A61K 47/10

(57) ABSTRACT

An aqueous ophthalmic composition is provided comprising at least one component which is selected from the group consisting of a polysaccharide, a monosaccharide, at least one vitamin component selected from the group consisting of vitamin B12, vitamin B2, vitamin A and panthenol, at least one oil component selected from the group consisting of a plant oil, an animal oil and a mineral oil, at least one surfactant selected from the group consisting of polyoxyethylene castor oil, polyoxyethylene hydrogenated castor oil, polyoxyethylene polyoxypropylene glycol and polyoxyl stearate, an anti-allergic component, a preservative, a thickening component, a polyhydric alcohol, an anti-inflammatory component, an anti-bacterial agent and a cooling and/or refreshing agent; and (B) a buffering agent. When the composition is brought into contact with a polybutylene terephthalate-containing resin container, the container can be prevented from weight change, deterioration and wetting and can be improved in a liquid cutting property.

### TECHNICAL FIELD

The present invention relates to an aqueous ophthalmic composition, a method for inhibiting a polybutylene terephthalate-containing resin container for ophthalmic use from being changed in weight, a method for inhibiting a polybutylene terephthalate-containing resin container for ophthalmic use from being deteriorated, and a method for inhibiting a polybutylene terephthalate-containing resin container for ophthalmic use from being wetted.

### BACKGROUND ART

A polybutylene terephthalate-containing resin which is one type of a thermoplastic polyester resin and which is generally used as a thermoplastic resin (which is referred to also as a PBT-containing resin in the present specification) has an excellent moldability and provides a good balance between the physical property and the price. Therefore, the resin is used as a container material for automobiles, electric and electronic components, semiconductor substrate containers, and the like.

Also, it is proposed to use a laminate body in which a PBT film and a thermally bonding resin layer are laminated as a packaging bag for heating that stores a food made of a liquid substance and a solid substance containing a large quantity of water, oil components, and sugar components inside thereof and that in particular does not generate holes even when subjected to cooking in a microwave oven or the like (Patent Document 1).

### PRIOR ART DOCUMENT

### Patent Document

Patent Document 1: JP-A-2006-143223

### SUMMARY OF THE INVENTION

### Problems to be Solved by the Invention

While the PBT-containing resin thus has an excellent property, when used as a container, it has a property of absorbing water in the contents stored in the container.

Furthermore, the PBT-containing resin hydrolyze by heat.

One of the objectives of the present invention is to provide an aqueous ophthalmic composition that can solve such problems.

### Means for Solving the Problems

As a result of repeated eager studies in order to solve the aforementioned problems, the present inventors have found out that, by incorporating specific components into an aqueous ophthalmic composition, a PBT-containing resin container that stores the composition is stabilized, and deterioration of the container can be inhibited, thereby completing the present invention. Also, the present inventors have found out that, by an aqueous ophthalmic composition containing a specific component, an improvement in terms of a wettability to the PBT-containing resin container can be made (that is, wetting can be inhibited), thereby producing a new effect of providing a good liquid-cutting property.

That is, the present invention provides:

An aqueous ophthalmic composition, comprising (A) at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil, and (B) a buffering agent, wherein (C) the aqueous ophthalmic composition is held in a container in which a part of a surface or the whole surface that is brought into contact with the aqueous ophthalmic composition is molded with a resin containing polybutylene terephthalate;

The aqueous ophthalmic composition according to Item [1], wherein, among the above (A) components, the polysaccharide is at least one selected from the group consisting of alginic acid, gellan gum, xanthan gum, hyaluronic acid, chondroitin sulfate, and salts thereof; the monosaccharide is glucose; the vitamin is at least one selected from the group consisting of cyanocobalamine, retinol, panthenol, flavin adenine dinucleotide, and salts thereof; the oil component is at least one selected from the group consisting of sesame oil, castor oil, lanolin, vaseline, and liquid paraffin; the surfactant is at least one selected from the group consisting of polyoxyethylene castor oil, polyoxyethylene hydrogenated castor oil, polyoxyethylene polyoxypropylene glycol, and polyoxyl stearate; the anti-allergic component is at least one selected from the group consisting of tranilast, ketotifen fumarate, and diphenhydramine hydrochloride; the preservative is at least one selected from the group consisting of chlorhexidine gluconate and potassium sorbate, the thickening component is at least one selected from the group consisting of carboxymethyl cellulose, methyl cellulose, polyvinylpyrrolidone, carboxyvinyl polymer, and salts thereof; the polyhydric alcohol is at least one selected from the group consisting of propylene glycol, glycerin, and mannitol; the anti-inflammatory component is at least one selected from the group consisting of berberine chloride, sodium azulenesulfonate, allantoin, and zinc sulfate; the anti-bacterial agent is sulfamethoxazole sodium; and the cooling and/or refreshing agent is selected from the group consisting of eucalyptus oil and bergamot oil;

The aqueous ophthalmic composition according to Item [1] or [2], further comprising sodium edetate;

A method for imparting a function of inhibiting change in weight of a polybutylene terephthalate-containing resin container to an aqueous composition by allowing (A) at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil, and (B) a buffering agent to coexist in the aqueous composition;

A method for imparting a function of inhibiting wetting of a polybutylene terephthalate-containing resin container to an aqueous composition by allowing (A) at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil, and (B) a buffering agent to coexist in the aqueous composition; and

An agent for improving a liquid-cutting property of a polybutylene terephthalate-containing resin container for ophthalmic use, comprising (A) at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil, and (B) a buffering agent.

Components and concentrations that will be described later may be used in the aqueous ophthalmic composition, the method for inhibiting change in weight of a polybutylene terephthalate-containing resin container, a method for inhibiting deterioration, and the method for inhibiting wetting, of the container, the agent for improving a liquid-cutting property, a method for improving a liquid-cutting property, and a production method according to the present invention.

### Effect of the Invention

When stored in a PBT-containing resin container, the aqueous ophthalmic composition according to the present invention stabilizes the container, and deterioration of the container can be prevented. Also, inhibition of wetting and improvement in the liquid-cutting property can be expected, and remaining of liquid in the container can be reduced.

### BRIEF DESCRIPTION OF THE DRAWING

FIG. 1 is a graph showing change in weight of a PBT-containing resin piece before and after immersing the PBT-containing resin piece into an aqueous ophthalmic composition and conducting a heat treatment.

### MODE FOR CARRYING OUT THE INVENTION

In the present invention, the unit “w/v %” of the content has the same significance as “g/100 mL”. In the present invention, the “blending amount” has the same significance as the “content”.

The present inventors have found out that, when an aqueous ophthalmic composition is stored in a PBT-containing resin container, change in weight of the PBT-containing resin occurs, resulting in problems such as decrease in the strength of the container, cracks, deformation, and decrease in the sealing property, that is, when a container formed of a PBT-containing resin is used in a mode in which a pharmaceutical agent such as an aqueous ophthalmic composition is used while being stored for a predetermined period of time, the problem of change in the property of the PBT-containing resin container becomes serious. The aqueous ophthalmic composition according to the present invention can solve problems such as these.

An aqueous ophthalmic composition according to the present invention contains (A) at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil, and (B) a buffering agent. The aqueous ophthalmic composition according to the present invention is stored in a PBT-containing resin container.

In the present invention, an aqueous composition refers to a composition containing water. The aqueous composition preferably contains 50 w/v % or more, more preferably 70 w/v % or more, further preferably 80 w/v % or more, still more preferably 85 w/v % or more, and particularly preferably 90 w/v % or more, of water relative to a total amount of the aqueous composition. In the present invention, an aqueous ophthalmic composition refers to all aqueous compositions related to ophthalmology such as eye drops (having the same significance as ophthalmic liquid or ophthalmic pharmaceutical agent), eye wash (having the same significance as eye-washing liquid or eye-washing pharmaceutical agent), liquid for wearing contact lenses, liquid for washing contact lenses, liquid for storing contact lenses, and disinfectant liquid for contact lenses.

In the aqueous ophthalmic composition of the present invention, it is possible to use, as the (A) component, at least one kind which is selected from the group consisting of a polysaccharide; a monosaccharide; at least one vitamin selected from the group consisting of vitamin B₁₂, vitamin B₂, vitamin A, and panthenol; at least one oil component selected from the group consisting of a plant oil, an animal oil, and a mineral oil; at least one surfactant selected from the group consisting of polyoxyethylene castor oil, polyoxyethylene hydrogenated castor oil, polyoxyethylene polyoxypropylene glycol, and polyoxyl stearate; at least one anti-allergic component selected from the group consisting of tranilast, ketotifen, diphenhydramine, and salts thereof; at least one preservative selected from the group consisting of chlorhexidine, sorbic acid, and salts thereof, at least one thickening component selected from the group consisting of carboxymethyl cellulose, methyl cellulose, a vinyl-based polymer compound, and salts thereof; a polyhydric alcohol; at least one anti-inflammatory component selected from the group consisting of berberine, azulenesulfonic acid, allantoin, zinc sulfate, and salts thereof; at least one anti-bacterial agent selected from the group consisting of sulfamethoxazole and salts thereof; and at least one cooling and/or refreshing agent selected from the group consisting of eucalyptus oil and bergamot oil.

In the present invention, these (A) components may be used either singly or in combination of two or more kinds. Those naturally obtained or chemically synthesized can also be used as the (A) components. Those commercially available can also be used for each of the (A) components.

In the present invention, the polysaccharide of the (A) component is preferably an acidic polysaccharide. The acidic polysaccharide refers to one containing a repeated structure of two or more kinds of monosaccharides and comprising an acidic group. Here, the acidic group is not limited; however, the acidic group particularly refers to a carboxyl group or a sulfuric acid group. The constituent components of the repeated structure are not limited, and examples thereof include uronic acids such as glucuronic acid, iduronic acid, mannuronic acid, and guluronic acid, amino sugars such as galactosamine and glucosamine, galactose, mannose, glucose, and rhamnose.

Such acidic polysaccharide is not limited, and examples thereof include hyaluronic acid, chondroitin sulfate, dermatan sulfate, heparin, keratan sulfate, xanthan gum, gellan gum, alginic acid, and salts thereof.

Those naturally obtained or chemically synthesized can also be used as the acidic polysaccharide of the (A) component, and the derivation is not particularly limited. Those commercially available can also be used for the acidic polysaccharide. The acidic polysaccharide may be used in a form of a salt of alkali metal such as sodium or potassium, a salt of alkaline earth metal such as calcium or magnesium, a salt of metal such as iron or manganese, or other physiologically or pharmaceutically acceptable salts. Also, an acetylated reaction product may be used. These acidic polysaccharides may be used either singly or in combination of two or more kinds. The acidic polysaccharide of the (A) component is preferably chondroitin sulfate, hyaluronic acid, xanthan gum, gellan gum, alginic acid, or a salt thereof, and is particularly preferably sodium chondroitin sulfate, sodium hyaluronate, alginic acid, or gellan gum.

The molecular weight of the acidic polysaccharide of the (A) component varies in accordance with the number of repeating units or the kind and is not limited; however, the molecular weight can be several hundred to several million in terms of weight-average molecular weight. From the viewpoint of producing the effects provided by the present invention more conspicuously such as inhibiting deterioration of the PBT-containing resin container, the molecular weight of the acidic polysaccharide of the (A) component is preferably 100 to 5,000,000, more preferably 500 to 3,000,000, in terms of weight-average molecular weight. More specifically, for example, the weight-average molecular weight of chondroitin sulfate or a salt thereof is preferably 1,000 to 3,000,000, more preferably 5,000 to 1,500,000, still more preferably 10,000 to 500,000. More specifically, for example, the weight-average molecular weight of hyaluronic acid or a salt thereof is preferably 100,000 to 5,000,000, more preferably 500,000 to 4,000,000, still more preferably 600,000 to 2,500,000.

In the present invention, examples of the monosaccharides of the (A) component include aldoses such as glucose (grape sugar), ribose, glyceraldehyde, erythrose, threose, lyxose, xylose, arabinose, allose, talose, gulose, altrose, mannose, galactose, and idose, and ketoses such as dihydroxyacetone, erythrulose, xylulose, ribulose, psicose, fructose, sorbose, and tagatose. Among these, glucose, galactose, mannose, fructose, and sorbose are preferable, and in particular, glucose is preferred. These may be used either singly or in combination of two or more kinds. Those commercially available can also be used as these monosaccharides.

In the present invention, the vitamin of the (A) component may be either a lipid-soluble vitamin or a water-soluble vitamin. The lipid-soluble vitamin may be, for example, at least one kind selected from the group consisting of vitamin A such as retinol, retinol acetate, retinol palmitate, retinal, retinoic acid, methyl retinoate, ethyl retinoate, retinol retinoate, fatty acid ester of vitamin A, d-δ-tocopheryl retinoate, α-tocopheryl retinoate, β-tocopheryl retinoate, carotin, dehydroretinal, lycopin, and salts thereof. The water-soluble vitamin may be, for example, at least one kind selected from the group consisting of vitamin B₂ such as riboflavin, flavin mononucleotide, flavin adenine dinucleotide, riboflavin butyrate, riboflavin tetrabutyrate, riboflavin 5′-phosphate sodium, riboflavin tetranicotinate, and salts thereof; vitamin B₁₂ such as cyanocobalamine, hydroxocobalamine, methylcobalamine, deoxyadenosylcobalamine, and salts thereof; and pantothenyl alcohol (panthenol).

The vitamin of the (A) component is preferably at least one kind selected from the group consisting of cyanocobalamine, retinol, panthenol, flavin adenine dinucleotide, and salts thereof, and is particularly preferably at least one kind selected from the group consisting of cyanocobalamine, retinol palmitate, retinol acetate, panthenol, and flavin adenine dinucleotide sodium. The vitamin A may be, for example, retinol palmitate manufactured by DSM N.V. in which 1 I.U. of vitamin A corresponds to 0.550 μg. Here, I.U. refers to an international unit as determined by a technique as described in The Japanese Pharmacopoeia Sixteenth Edition Vitamin A Assay or the like.

These vitamins may be used either singly or in combination of two or more kinds. Those commercially available can also be used as any of these vitamins.

In the present invention, the oil component of the (A) component may be at least one oil component selected from the group consisting of a plant oil, an animal oil, and a mineral oil. When an oil component is used as the (A) component, the oil component is preferably a plant oil and/or a mineral oil from the viewpoint of producing the effects provided by the present invention more conspicuously.

Here, the plant oil is not particularly limited as long as the plant oil is an oil produced by using a plant as a source material. A plant oil containing triglyceride is preferable. Although not limited, specific examples of the plant oil of the (A) component include sesame oil, castor oil, soybean oil, peanut oil, almond oil, wheat germ oil, camellia oil, corn oil, rapeseed oil, sunflower oil, cotton-seed oil, olive oil, and derivatives thereof. The plant oil of the (A) component is preferably sesame oil, castor oil, soybean oil, or a derivative thereof, and is particularly preferably sesame oil or castor oil. These plant oils may be used either singly or in combination of two or more kinds. Those commercially available can also be used as any of these plant oils.

Although not limited, specific examples of the animal oil of the (A) component include squalane, lanolin, orange roughy oil, horse oil, whale oil, liver oil, mink oil, yolk oil, beef tallow, milk fat, and pig oil. The animal oil of the (A) component is preferably squalane, lanolin, yolk oil, or a derivative thereof, and is particularly preferably squalane or refined lanolin. Here, the animal oil is not particularly limited as long as the animal oil is an oil produced by using an animal as a source material. These animal oils may be used either singly or in combination of two or more kinds. Those commercially available may also be used as any of these animal oils.

The mineral oil of the (A) component refers to a chemical substance in a liquid form or a grease form obtained by refining a hydrocarbon oil derived from natural petroleum. Although not limited, specific examples of the mineral oil of the (A) component include paraffin oil, liquid paraffin, and vaseline. The mineral oil of the (A) component is particularly preferably liquid paraffin, light liquid paraffin, or white vaseline. These mineral oils may be used either singly or in combination of two or more kinds. Those commercially available can also be used as any of these mineral oils. For example, HICALL M-202 manufactured by Kaneda Co., Ltd. and the like can be mentioned as an example of the liquid paraffin.

In the present invention, the surfactant of the (A) component may be at least one kind selected from the group consisting of polyoxyethylene castor oil, polyoxyethylene hydrogenated castor oil, polyoxyethylene polyoxypropylene glycol, and polyoxyl stearate.

Specific examples of the surfactant include polyoxyethylene polyoxypropylene glycols such as Poloxamer 407, polyoxyethylene (200) polyoxypropylene (70) glycol, Poloxamer 188, polyoxyethylene (120) polyoxypropylene (40) glycol, polyoxyethylene (54) polyoxypropylene (39) glycol, polyoxyethylene (20) polyoxypropylene (20) glycol, and Tetronic; polyoxyethylene hydrogenated castor oils such as polyoxyethylene hydrogenated castor oil 5, polyoxyethylene hydrogenated castor oil 10, polyoxyethylene hydrogenated castor oil 20, polyoxyethylene hydrogenated castor oil 40, polyoxyethylene hydrogenated castor oil 50, polyoxyethylene hydrogenated castor oil 60, and polyoxyethylene hydrogenated castor oil 100; polyoxyethylene castor oils such as polyoxyethylene castor oil 3, polyoxyethylene castor oil 4, polyoxyethylene castor oil 6, polyoxyethylene castor oil 7, polyoxyethylene castor oil 10, polyoxyethylene castor oil 13.5, polyoxyethylene castor oil 17, polyoxyethylene castor oil 20, polyoxyethylene castor oil 25, polyoxyethylene castor oil 35, polyoxyethylene castor oil 40, polyoxyethylene castor oil 50, and polyoxyethylene castor oil 60; and polyoxyl stearates such as polyoxyl 40 stearate and polyoxyl 140 stearate.

Among the surfactants of the (A) component, Poloxamer 407, polyoxyethylene (200) polyoxypropylene (70) glycol, polyoxyethylene hydrogenated castor oil 40, polyoxyethylene hydrogenated castor oil 60, polyoxyethylene castor oil 3, polyoxyethylene castor oil 10, polyoxyethylene castor oil 35, polyoxyl 40 stearate and polyoxyl 140 stearate are preferable, and Poloxamer 407, polyoxyethylene hydrogenated castor oil 60, polyoxyethylene castor oil 10, polyoxyethylene castor oil 35, and polyoxyl 40 stearate are more preferable.

An average number of moles of added ethylene oxide in the polyoxyethylene castor oil used as the (A) component is not particularly limited; however, the average number can be set to be, for example, 2 to 70 mol, preferably 2 to 60, more preferably 3 to 50, and particularly preferably 3 to 40. An average number of moles of added ethylene oxide in the polyoxyethylene polyoxypropylene glycol used as the (A) component is not particularly limited; however, the average number can be set to be, for example, 10 to 350 mol, preferably 30 to 300, more preferably 50 to 300, and particularly preferably 100 to 250. An average number of moles of added ethylene oxide in the polyoxyethylene hydrogenated castor oil used as the (A) component is not particularly limited; however, the average number can be set to be, for example, 3 to 120 mol, preferably 20 to 100, and more preferably 30 to 80. An average number of moles of added ethylene oxide in the polyoxyl stearate used as the (A) component is not particularly limited; however, the average number can be set to be, for example, 3 to 200 mol, preferably 20 to 180, and more preferably 30 to 160.

In the present invention, the (A) component may be at least one anti-allergic component selected from the group consisting of tranilast, ketotifen, diphenhydramine, and salts thereof. Among the anti-allergic components, tranilast, ketotifen fumarate, and diphenhydramine hydrochloride are preferable.

In the present invention, the (A) component may be at least one preservative selected from the group consisting of chlorhexidine, sorbic acid, and salts thereof. Among the preservatives, chlorhexidine gluconate, sorbic acid, and potassium sorbate are preferable.

In the present invention, the thickening component of the (A) component may be either a cellulose-based polymer compound or a vinyl-based polymer compound. Although not limited, examples of the cellulose-based polymer compounds include carboxymethyl cellulose, methyl cellulose, and salts thereof. Although not limited, examples of the vinyl-based polymer compounds include polyvinylpyrrolidone, polyvinyl alcohol (fully or partially saponified product), carboxyvinyl polymer, and salts thereof. As the thickening component of the (A) component, carboxymethyl cellulose, methyl cellulose, vinyl-based polymer compounds, and salts thereof are preferable. Among these, carboxymethyl cellulose, carboxymethyl cellulose sodium, methyl cellulose, polyvinylpyrrolidone, and carboxyvinyl polymer are more preferable. Among these, carboxymethyl cellulose, carboxymethyl cellulose sodium, methyl cellulose, polyvinylpyrrolidone K17, polyvinylpyrrolidone K25, polyvinylpyrrolidone K30, polyvinylpyrrolidone K90, and carboxyvinyl polymer are still more preferable. Among these, carboxymethyl cellulose, carboxymethyl cellulose sodium, methyl cellulose, polyvinylpyrrolidone K25, polyvinylpyrrolidone K30, polyvinylpyrrolidone K90, and carboxyvinyl polymer are still more preferable.

In the present invention, the (A) component can be a polyhydric alcohol. Although not limited, the polyhydric alcohol is preferably at least one kind selected from the group consisting of propylene glycol, glycerin, and mannitol.

In the present invention, the (A) component may be at least one anti-inflammatory component selected from the group consisting of berberine, azulenesulfonic acid, allantoin, zinc sulfate, and salts thereof. Among the anti-inflammatory components, berberine sulfate, berberine chloride, sodium azulenesulfonate, allantoin, and zinc sulfate are preferable.

In the present invention, the (A) component may be at least one anti-bacterial agent selected from the group consisting of sulfamethoxazole and salts thereof. Here, the salt of sulfamethoxazole is preferably sulfamethoxazole sodium.

In the present invention, the (A) component may be at least one cooling and/or refreshing agent selected from the group consisting of eucalyptus oil and bergamot oil.

These (A) components all may be used either singly or in any combination of two or more kinds. In particular, two or more kinds are preferably combined. When two or more kinds are combined, for example, two or more kinds of different substances belonging to the same classification can be combined, or alternatively, two or more kinds of different substances belonging to different classifications can be combined. For example, two or more kinds of polysaccharides can be incorporated, or alternatively, at least one polysaccharide and at least one vitamin can be selected and combined. The same applies to the (A) components such as the monosaccharide, the oil component, the surfactant, the anti-allergic component, the preservative, the thickening component, the polyhydric alcohol, the anti-inflammatory component, the anti-bacterial agent, and the cooling and/or refreshing agent.

In the aqueous ophthalmic composition of the present invention, from the viewpoint of producing the effects provided by the present invention more conspicuously, the total content of the (A) components relative to the total amount of the aqueous ophthalmic composition is suitably set in accordance with the kinds of the (A) components and the kinds and content of other blended components. The total content of the (A) components is preferably 0.0001 w/v % or more, more preferably 0.001 w/v % or more, more preferably 0.005 w/v % or more, and still more preferably 0.01 w/v % or more, relative to the total amount of the aqueous ophthalmic composition. The total content of the (A) components is preferably 20 w/v % or less, more preferably 10 w/v % or less, still more preferably 5 w/v % or less, more preferably 3 w/v % or less, and most preferably 1 w/v % or less, relative to the total amount of the aqueous ophthalmic composition.

The total content of the polysaccharide relative to the total amount of the aqueous ophthalmic composition of the present invention is suitably set in accordance with the kinds of the (A) components and the kinds and content of other blended components. From the viewpoint of producing the effects provided by the present invention more conspicuously, the total content of the polysaccharide is preferably from 0.0001 w/v % to 6 w/v %, more preferably from 0.0005 w/v % to 4 w/v %, and particularly preferably from 0.001 w/v % to 2 w/v %, relative to the total amount of the aqueous ophthalmic composition.

Although not limited, in a preferable embodiment, for example, when chondroitin sulfate or a salt thereof is contained as the (A) component, the content of chondroitin sulfate or a salt thereof alone is preferably from 0.0001 w/v % to 5 w/v %, more preferably from 0.005 w/v % to 3 w/v %, relative to the total amount of the aqueous ophthalmic composition. Similarly, in another preferable embodiment, for example, when hyaluronic acid or a salt thereof is contained as the (A) component, the content of hyaluronic acid or a salt thereof alone is preferably from 0.0001 w/v % to 1 w/v %, more preferably from 0.0005 w/v % to 0.5 w/v %, relative to the total amount of the aqueous ophthalmic composition.

The total content of the monosaccharide relative to the total amount of the aqueous ophthalmic composition of the present invention is suitably set in accordance with the kinds of the (A) components and the kinds and content of other blended components. From the viewpoint of producing the effects provided by the present invention more conspicuously, the total content of the monosaccharide is preferably from 0.0001 w/v % to 3 w/v %, more preferably from 0.005 w/v % to 1.5 w/v %, and particularly preferably from 0.001 w/v % to 0.5 w/v %, relative to the total amount of the aqueous ophthalmic composition.

In another preferable embodiment, when glucose is contained as the (A) component, the content of glucose alone is preferably from 0.0001 w/v % to 3 w/v %, more preferably from 0.005 w/v % to 1.5 w/v %, and particularly preferably from 0.001 w/v % to 0.5 w/v %, relative to the total amount of the aqueous ophthalmic composition.

The total content of the vitamin relative to the total amount of the aqueous ophthalmic composition of the present invention is suitably set in accordance with the kinds of the (A) components and the kinds and content of other blended components. From the viewpoint of producing the effects provided by the present invention more conspicuously, the total content of the vitamin is preferably from 0.00001 w/v % to 1.6 w/v %, more preferably from 0.0005 w/v % to 0.8 w/v %, and particularly preferably from 0.0005 w/v % to 0.4 w/v %, relative to the total amount of the aqueous ophthalmic composition.

Although not limited, in a preferable embodiment, for example, when retinol palmitate is contained as the (A) component, the content of retinol palmitate alone is preferably from 10 to 500,000 units/100 mL, more preferably from 100 to 300,000 units/100 mL, and still more preferably from 500 to 200,000 units/100 mL, of the total amount of the aqueous ophthalmic composition. Though depending on the unit of blended retinol palmitate, the content is preferably from 0.005 to 0.5 W/V %, more preferably from 0.001 to 0.4 W/V %, and more preferably from 0.01 to 0.3 W/V %. Similarly, in another preferable embodiment, for example, cyanocobalamine as a single (A) component is contained preferably at 0.00001 w/v % to 1 w/v %, more preferably at 0.00005 w/v % to 0.5 w/v %, and particularly preferably from 0.0001 w/v % to 0.02 w/v %, relative to the total amount of the aqueous ophthalmic composition.

The total content of the oil component relative to the total amount of the aqueous ophthalmic composition of the present invention is suitably set in accordance with the kinds of the (A) components and the kinds and content of other blended components. From the viewpoint of producing the effects provided by the present invention more conspicuously, the total content of the oil component is preferably from 0.00001 w/v % to 6 w/v %, more preferably from 0.0005 w/v % to 3 w/v %, and particularly preferably from 0.0001 w/v % to 1 w/v %, relative to the total amount of the aqueous ophthalmic composition.

Although not limited, in a preferable embodiment, for example, when sesame oil is contained as the (A) component, the content of sesame oil alone is preferably from 0.00001 w/v % to 5 w/v %, more preferably from 0.0001 w/v % to 1 w/v %, relative to the total amount of the aqueous ophthalmic composition. Similarly, in another preferable embodiment, for example, when castor oil is contained as the (A) component, the content of castor oil alone is preferably from 0.00001 w/v % to 5 w/v %, more preferably from 0.0001 w/v % to 1 w/v %, relative to the total amount of the aqueous ophthalmic composition. Similarly, in another preferable embodiment, for example, when liquid paraffin is contained as the (A) component, the content of liquid paraffin alone is preferably from 0.00001 w/v % to 2 w/v %, more preferably from 0.0001 w/v % to 1 w/v %, relative to the total amount of the aqueous ophthalmic composition. Similarly, in another preferable embodiment, for example, when vaseline is contained as the (A) component, the content of vaseline alone is preferably from 0.00001 w/v % to 5 w/v %, more preferably from 0.00005 w/v % to 1 w/v %, relative to the total amount of the aqueous ophthalmic composition.

The total content of the surfactant relative to the total amount of the aqueous ophthalmic composition of the present invention is suitably set in accordance with the kinds of the (A) components and the kinds and content of other blended components. From the viewpoint of producing the effects provided by the present invention more conspicuously, the total content of the surfactant is preferably from 0.00001 w/v % to 10 w/v %, more preferably from 0.0001 w/v % to 8 w/v %, and particularly preferably from 0.001 w/v % to 5 w/v %, relative to the total amount of the aqueous ophthalmic composition.

Although not limited, in a preferable embodiment, for example, when polyoxyethylene polyoxypropylene glycol is contained as the (A) component, the content of polyoxyethylene polyoxypropylene glycol alone is preferably from 0.00001 w/v % to 10 w/v %, more preferably from 0.0001 w/v % to 8 w/v %, and particularly preferably from 0.001 w/v % to 5 w/v %, relative to the total amount of the aqueous ophthalmic composition. Similarly, in another preferable embodiment, for example, when polyoxyethylene castor oil is contained as the (A) component, the content of polyoxyethylene castor oil alone is preferably from 0.00001 w/v % to 10 w/v %, more preferably from 0.0001 w/v % to 5 w/v %, and particularly preferably from 0.001 w/v % to 3 w/v %, relative to the total amount of the aqueous ophthalmic composition.

From the viewpoint of producing the effects provided by the present invention more conspicuously, the total content of the anti-allergic component relative to the total amount of the aqueous ophthalmic composition of the present invention is preferably from 0.00001 w/v % to 5 w/v %, more preferably from 0.0005 w/v % to 1 w/v %, and particularly preferably from 0.0005 w/v % to 0.5 w/v %, relative to the total amount of the aqueous ophthalmic composition.

From the viewpoint of producing the effects provided by the present invention more conspicuously, the total content of the preservative relative to the total amount of the aqueous ophthalmic composition of the present invention is preferably from 0.00001 w/v % to 2 w/v %, more preferably from 0.00005 w/v % to 1 w/v %, and particularly preferably from 0.0001 w/v % to 0.5 w/v %, relative to the total amount of the aqueous ophthalmic composition.

From the viewpoint of producing the effects provided by the present invention more conspicuously, the total content of the thickening component relative to the total amount of the aqueous ophthalmic composition of the present invention is preferably from 0.0001 w/v % to 10 w/v %, more preferably from 0.0005 w/v % to 8 w/v %, and particularly preferably from 0.001 w/v % to 5 w/v %, relative to the total amount of the aqueous ophthalmic composition.

From the viewpoint of producing the effects provided by the present invention more conspicuously, the total content of the polyhydric alcohol relative to the total amount of the aqueous ophthalmic composition of the present invention is preferably from 0.00005 w/v % to 10 w/v %, more preferably from 0.0001 w/v % to 8 w/v %, and particularly preferably from 0.005 w/v % to 5 w/v %, relative to the total amount of the aqueous ophthalmic composition.

From the viewpoint of producing the effects provided by the present invention more conspicuously, the total content of the anti-inflammatory component relative to the total amount of the aqueous ophthalmic composition of the present invention is preferably from 0.00001 w/v % to 3 w/v %, more preferably from 0.00005 w/v % to 1.5 w/v %, and particularly preferably from 0.0001 w/v % to 0.6 w/v %, relative to the total amount of the aqueous ophthalmic composition.

From the viewpoint of producing the effects provided by the present invention more conspicuously, the total content of the anti-bacterial component relative to the total amount of the aqueous ophthalmic composition of the present invention is preferably from 0.01 w/v % to 6 w/v %, more preferably from 0.05 w/v % to 5 w/v %, and particularly preferably from 0.4 w/v % to 4 w/v %, relative to the total amount of the aqueous ophthalmic composition.

From the viewpoint of producing the effects provided by the present invention more conspicuously, the total content of the cooling and/or refreshing agent relative to the total amount of the aqueous ophthalmic composition of the present invention is preferably from 0.0001 w/v % to 1 w/v %, more preferably from 0.0005 w/v % to 0.5 w/v %, and particularly preferably from 0.001 w/v % to 0.1 w/v %, relative to the total amount of the aqueous ophthalmic composition.

In the present invention, the buffering agent of the (B) component may be either an inorganic buffering agent or an organic buffering agent.

The inorganic buffering agent of the (B) component of the present invention is preferably boric acid or a salt of boric acid. The salt of boric acid is not particularly limited as long as the salt of boric acid is a physiologically or pharmaceutically acceptable salt. An alkali metal salt, an alkaline earth metal salt, and a salt with an organic base, of boric acid are exemplified. In more detail, a salt of boric acid with sodium, potassium, calcium, magnesium, ammonium, diethanolamine, ethylenediamine, or the like can be mentioned as an example. Although not limited, specific preferable examples of the boric acid salts include borax, sodium borate, ammonium borate, and potassium tetraborate. Among these, borax is particularly preferably used.

The organic buffering agent of the (B) component of the present invention is preferably epsilon-aminocaproic acid, phosphoric acid, citric acid, carbonic acid, 2-amino-2-hydroxymethyl-1,3-propanediol (Tris, trometamol, trishydroxymethylaminomethane), or a salt thereof. These salts also are not particularly limited as long as these are physiologically or pharmaceutically acceptable salts. An alkali metal salt, an alkaline earth metal salt, and a salt with an organic base, of epsilon-aminocaproic acid, phosphoric acid, citric acid, carbonic acid, or 2-amino-2-hydroxymethyl-1,3-propanediol are exemplified. Preferably, a salt with sodium, potassium, calcium, magnesium, ammonium, diethanolamine, ethylenediamine, or the like can be mentioned as an example.

In the present invention, these (B) components may be used either singly or in combination of two or more kinds. Those naturally obtained or chemically synthesized can also be used as the (B) components. Those commercially available can also be used for each of the (B) components.

In the aqueous ophthalmic composition of the present invention, from the viewpoint of producing the effects provided by the present invention more conspicuously, the total content of the (B) components is preferably 0.001 w/v % or more, more preferably 0.01 w/v % or more, and still more preferably 0.1 w/v % or more, relative to the total amount of the aqueous ophthalmic composition. Also, the total content of the (B) components is preferably 20 w/v % or less, more preferably 15 w/v % or less, still more preferably 10 w/v % or less, more preferably 5 w/v % or less, and most preferably 3 w/v % or less, relative to the total amount of the aqueous ophthalmic composition.

Although not limited, in a preferable embodiment, for example, when epsilon-aminocaproic acid or 2-amino-2-hydroxymethyl-1,3-propanediol is contained as the (B) component, the content of epsilon-aminocaproic acid or 2-amino-2-hydroxymethyl-1,3-propanediol alone is preferably from 0.001 w/v % to 6 w/v %, more preferably from 0.01 w/v % to 8 w/v %, and particularly preferably from 0.05 w/v % to 5 w/v %, relative to the total amount of the aqueous ophthalmic composition. Similarly, in another preferable embodiment, for example, when boric acid, phosphoric acid, citric acid, carbonic acid, or a salt thereof is contained as the (B) component, the content of boric acid, phosphoric acid, citric acid, carbonic acid, or a salt thereof alone is preferably from 0.001 w/v % to 5 w/v %, more preferably from 0.005 w/v % to 4 w/v %, and particularly preferably from 0.01 w/v % to 3 w/v %, relative to the total amount of the aqueous ophthalmic composition.

In the aqueous ophthalmic composition of the present invention, from the viewpoint of producing the effects provided by the present invention more conspicuously, a ratio of the content of the (B) components relative to the (A) components is preferably as follows. That is, the total content of the (B) components is preferably from 0.00001 to 10000 parts by weight, more preferably from 0.0001 to 5000 parts by weight, still more preferably from 0.0005 to 3000 parts by weight, particularly preferably from 0.001 to 2000 parts by weight, and most preferably from 0.01 to 1000 parts by weight, relative to 1 part by weight of the total content of the (A) components.

In the aqueous ophthalmic composition of the present invention, a combination of the (A) component and the (B) component is not particularly limited, and is suitably set in accordance with the kinds of the (A) component and the (B) component and the like. Combinations are exemplified in the following Table 1 that extends in two pages.

TABLE 1

| | (A) Component | (B) Component |
|---|---|---|
| 1 | Sodium chondroitin sulfate | Boric acid |
| 2 | Sodium chondroitin sulfate | Citric acid |
| 3 | Sodium chondroitin sulfate | Disodium hydrogen phosphate |
| 4 | Sodium chondroitin sulfate | 2-amino-2-hydroxymethyl-1,3-propanediol |
| 5 | Sodium chondroitin sulfate | Epsilon-aminocaproic acid |
| 6 | Sodium hyaluronate | Epsilon-aminocaproic acid |
| 7 | Sodium hyaluronate | Disodium hydrogen phosphate |
| 8 | Sodium hyaluronate | Boric acid |
| 9 | Xanthan gum | Boric acid |
| 10 | Gellan gum | Boric acid |
| 11 | Alginic acid | Boric acid |
| 12 | Retinol palmitate | Boric acid |
| 13 | Retinol palmitate | Boric acid and |
| | | 2-amino-2-hydroxymethyl-1,3-propanediol |
| 14 | Glucose | Boric acid |
| 15 | Cyanocobalamine | Boric acid |
| 16 | Cyanocobalamlne | Epsilon-aminocaproic acid. |
| 17 | Flavin adenine dinucleotide sodium | Boric acid |
| 18 | Flavin adenine dinucleotide sodium | Epsilon-aminocaproic acid |
| 19 | Panthenol | Boric acid |
| 20 | Sesame oil | Boric acid |
| 21 | Sesame oil | 2-amino-2-hydroxymethyl-1,3-propanediol |
| 22 | Sesame oil | Epsilon-aminocaproic acid |
| 23 | Sesame oil | Disodium hydrogen phosphate |
| 24 | Castor oil | Boric acid |
| 25 | Refined lanolin | Boric acid |
| 26 | White vaseline | Boric acid |
| 27 | Liquid paraffin | Boric acid |
| 28 | Polyoxyethylene castor oil 10 | Boric acid |
| 29 | Polyoxyethylene castor oii 3 | Boric acid |
| 30 | Polyoxyethylene (200) polyoxypropylene (70) | Boric acid |
| | glycol | |
| 31 | Polyoxyethylene (196) polyoxypropylene (67) | Boric acid |
| | glycol | |
| 32 | Polyoxyethylene (200) polyoxypropylene (70) | 2-amino-2-hydroxymethyl-1,3-propanediol |
| | glycol. | |
| 33 | Polyoxyethylene (196) polyoxypropylene (67) | 2-amino-2-hydroxymethyl-1,3-propanediol |
| | glycol | |
| 34 | Polyoxyethylene hydrogenated castor oil 60 | Boric acid |
| 35 | Polyoxyethylene hydrogenated castor oil 40 | Boric acid |
| 36 | Polyoxyethylene hydrogenated castor oil 60 | Disodium hydrogen phosphate |
| 37 | Polyoxyethylene hydrogenated castor oil 60 | 2-amino-2-hydroxymethyl-1,3-propanediol |
| 38 | Polyoxyethylene hydrogenated castor oii 60 | Epsilon-aminocaproic acid |
| 39 | Polyoxyethylene hydrogenated castor oil 60 | Citric acid |
| 40 | Polyoxyethylene hydrogenated castor oil 60 | Carbonic acid |
| 41 | Polyoxyl 40 stearate | Boric acid |
| 42 | Polyoxyl 40 stearate | Disodium hydrogen phosphate |
| 43 | Tranilast | Boric acid |
| 44 | Ketotifen | Boric acid |
| 45 | Diphenhydramine hydrochloride | Boric acid |
| 46 | Chlorhexidine gluconate | Boric acid |
| 47 | Potassium sorbate | Boric acid |
| 48 | Carboxymethyl cellulose sodium | Boric acid |
| 49 | Methyl cellulose | Boric acid |
| 50 | Methyl cellulose | Disodium hydrogen phosphate |
| 51 | Polyvinyl pyrrolidone | Boric acid |
| 52 | Polyvinyl pyrrolidone | Disodium hydrogen phosphate |
| 53 | Polyvinyl pyrrolidone | 2-amino-2-hydroxymethyl-1,3-propanediol |
| 54 | Polyvinyl pyrrolidone | Epsilon-aminocaproic acid |
| 55 | Carboxyvinyl polymer | Boric acid |
| 56 | Carboxyvinyl polymer | Epsilon-aminocaproic acid |
| 57 | Propylene glycol | Boric acid |
| 58 | Propylene glycol | Epsilon-aminocaproic acid |
| 59 | Propylene glycol | Disodium hydrogen phosphate |
| 60 | Propylene glycol | 2-amino-2-hydroxymethyl-1,3-propanediol |
| 61 | Glycerin | Boric acid |
| 62 | Glycerin | Disodium hydrogen phosphate |
| 63 | Glycerin | 2-amino-2-hydroxymethyl-1,3-propanediol |
| 64 | Glycerin | Epsilun-aminocaproic acid |
| 65 | Mannitol | Boric acid |
| 66 | Mannitol | 2-amino-2-hydroxymethyl-1,3-propanediol |
| 67 | Berberine sulfate | Boric acid |
| 68 | Sodium azulenesulfonate | Boric acid |
| 69 | Allantoin | Boric acid |
| 70 | Zinc sulfate | Boric acid |
| 71 | Sulfamethoxazole sodium | Boric acid |
| 72 | Sulfamethoxazole | 2-amino-2-hydroxymethyl-1,3-propanediol |
| 73 | Sulfamethoxazole | Epsilon-aminocaproic acid |
| 74 | Sulfamethoxazole | Borax |
| 75 | Bergamot oil | Boric acid |
| 76 | Eucalyptus oil | Boric acid |

In the present invention, the PBT-containing resin container refers to a container for ophthalmic use in which a part of the container or the whole container is molded with a resin containing polybutylene terephthalate. Here, the “part of the container” refers to at least one part of the portion that is brought into contact with an aqueous ophthalmic composition that is stored in the inside. The portion that is brought into contact with the aqueous ophthalmic composition can be an inside plug, a perforated inside plug, an innermost layer in a structure made of a plurality of layers constructed on an inner surface of the container, or the like. For example, a container having a perforated inside plug (nozzle) may have a structure in which only the inside plug portion is formed of a PBT-containing resin. Alternatively, a structure in which a storage portion or the like other than the inside plug is formed of a PBT-containing resin. Alternatively, a structure in which the whole container is molded with a PBT-containing resin. It is sufficient that at least a part of the surface that is brought into contact with the aqueous ophthalmic composition is formed of a PBT-containing resin; however, it is most preferable that the whole contact surface is formed of a PBT-containing resin. When a part of the container is formed of a PBT-containing resin, the kind of the resin that forms the other parts is not particularly limited; however, the container may contain at least one polymer selected from the group consisting of polyethylene terephthalate (PET), polystyrene (PS), acrylonitrile butadiene styrene (ABS), polycarbonate, polyethylene (PE), polypropylene (PP), polymethyl methacrylate, ethylene vinyl acetate copolymer, and ethylene vinyl alcohol copolymer as a constituent component.

In the present invention, the shape of the PBT-containing resin container and the volume that can be stored in the inside are not particularly limited. For example, when the PBT-containing resin container is for ordinary eye drops or a liquid for wearing contact lenses, the container can be a container capable of storing 0.1 ml or more and 50 ml or less, preferably 2 ml or more and 40 ml or less, more preferably 4 ml or more and 25 ml or less, of the contents. When the PBT-containing resin container is for an eye wash or a liquid for contact lens care, the volume that can be stored in the inside can be 40 ml or more and 600 ml or less.

Besides this, the PBT-containing resin container of the present invention can be a container capable of storing an aqueous ophthalmic composition that is applied to contact lenses.

The aqueous ophthalmic composition that is used in the present invention may be either a multi-dose type in which an amount for use of plural times is stored or a unit-dose type in which an amount for use of a single time is stored.

In particular, in the present invention, the PBT-containing resin container is preferably an eye-drop container, an eye-wash container, a container for storing liquid for wearing contact lenses, a container for storing liquid for contact lens care (including a container for storing contact lens washing liquid, a container for storing contact lens storing liquid, a container for storing contact lens disinfectant liquid, a container for storing contact lens multi-purpose solution, and the like), or a container for storing liquid for packaging contact lenses. The PBT-containing resin container is particularly preferably an eye-drop container, a container for storing liquid for wearing contact lenses, or a container for storing liquid for contact lens care. Here, the contact lenses as described herein refer to any kind of contact lenses, and may be either soft contact lenses or hard contact lenses.

The present invention also provides a product in a state in which an aqueous ophthalmic composition is stored in a PBT-containing resin container. The present invention also provides eye drops, an eye wash, and a contact-applied product of a container in which an aqueous ophthalmic composition is stored.

The PBT-containing resins in the PBT-containing resin containers of the present invention include polymers obtained by a known polymerization method such as condensation polymerization of terephthalic acid or an ester-forming derivative thereof with 1,4-butanediol. These polymers can be formed into PBT-containing resins by adding an additive such as a stabilizer. PBT-containing resins commercially available as PBT-containing resins may be used without any particular limitation. An example thereof may be “NOVADURAN (registered trademark) 5010R5” manufactured by Mitsubishi Engineering-Plastics Corporation or the like. The polymer synthesized by condensation polymerization of terephthalic acid or an ester-forming derivative thereof with 1,4-butanediol may contain other monomers arbitrarily as constituent components, and may further contain other polymers. Examples of the other polymers include polycarbonate, (meth)acrylic acid-based polymers, polystyrene (PS), polyethylene naphthalate (PEN), polyethylene terephthalate (PET), polyethylene (PE), polyarylate, and polypropylene (PP); however, the other polymers are not limited thereto. Here, although not limited, dimethyl terephthalate and the like can be exemplified as the ester-forming derivative of terephthalic acid. Preferably, in the PBT-containing resin of the present invention, the polymer synthesized by condensation polymerization of terephthalic acid or an ester-forming derivative thereof with 1,4-butanediol occupies 50 wt % or more, more preferably 60 wt % or more, and still more preferably 70 wt % or more, in the polymer components constituting the resin. Those commercially available can also be used as these polymers.

The PBT-containing resins of the present invention further include resins reinforced by containing a reinforcing agent such as a glass fiber.

In the aqueous ophthalmic composition of the present invention, it is preferable to incorporate other components that can be generally used in the aqueous ophthalmic compositions in addition to the (A) components and the (B) components. Such components are not particularly limited; however, from the viewpoint of producing the effects provided by the present invention more conspicuously, a particularly preferable example thereof can be sodium edetate. In the present invention, commercially available sodium edetate may be used as well.

In the aqueous ophthalmic composition of the present invention, from the viewpoint of producing the effects provided by the present invention more conspicuously, the total content of sodium edetate is preferably 0.0001 w/v % or more, more preferably 0.0005 w/v % or more, and still more preferably 0.001 w/v % or more, relative to the total amount of the aqueous ophthalmic composition. The total content of sodium edetate is preferably 1 w/v % or less, more preferably 0.5 w/v % or less, and still more preferably 0.2 w/v % or less, relative to the total amount of the aqueous ophthalmic composition. For the ratio of the content of sodium edetate relative to the (A) components, the total content of sodium edetate is preferably from 0.0001 to 1000 parts by weight, more preferably from 0.0005 to 500 parts by weight, and still more preferably from 0.001 to 200 parts by weight, relative to 1 part by weight of the total content of the (A) components.

In addition, active ingredients in pharmaceutical agents for ophthalmic use described in Manufacturing (Import) Standard to Approval of Medical Supplies for Public 2012 Edition (edited by Society for Regulatory Science of Medical Products, a general incorporated association) can be exemplified. Specific examples thereof may be the following ingredients.

Anti-histamic agents: chlorpheniramine maleate

Anti-allergic agents: acitazanolast, amlexanox, ibudilast, levocabastine hydrochloride, sodium cromoglicate, pemirolast potassium, olopatadine hydrochloride, and others.

Decongestants: tetrahydrozoline hydrochloride, naphazoline hydrochloride, naphazoline nitrate, epinephrine, epinephrine hydrochloride, ephedrine hydrochloride, phenylephrine hydrochloride, methylephedrine hydrochloride, and others.

Amino acids: potassium aspartate, magnesium aspartate, aminoethylsulfonic acid, and others.

Anti-inflammatory agents: dipotassium glycyrrhizinate, lysozyme chloride, pranoprofen, bromfenac, ketrolac tromethamine, nepafenac, and others.

Astringents: zinc white, zinc lactate, and others.

Others: sulfisoxazole, sulfisomidine sodium, neostigmine methylsulfate, dibucaine, and others.

Furthermore, in the aqueous ophthalmic composition of the present invention, additives such as a carrier, a thickening agent, a pH regulator, a general sugar, a general tonicifier, a flavoring agent, a cooling and/or refreshing agent, and a chelating agent may be selected, and a suitable amount may be incorporated by using at least one kind in combination. As these additives, various kinds of additives described in Japanese Pharmaceutical Excipients Directory 2007 (edited by Japanese Pharmaceutical Excipients Council) can be exemplified. Representative ingredients may be the following additives.

Carriers: aqueous carriers such as water and water-containing ethanol.

Thickening agents: hydroxypropyl cellulose, hydroxypropyl methyl cellulose, hydroxyethyl cellulose, and others.

Sugar alcohols: xylitol, sorbitol, and others. These may be in any of a d-form, an l-form, and a dl-form.

Tonicifiers: aminoethylsulfonic acid, polyethylene glycol, sodium hydrogen sulfite, sodium sulfite, potassium chloride, calcium chloride, sodium chloride, magnesium chloride, and others.

pH adjusters: hydrochloric acid, acetic acid, sodium hydroxide, potassium hydroxide, calcium hydroxide, magnesium hydroxide, triethanolamine, monoethanolamine, diisopropanolamine, and others.

Stabilizers: dibutylhydroxytoluene, sodium formaldehyde sulfoxylate (Rongalit), sodium hydrogen sulfite, sodium pyrosulfite, monoethanolamine, aluminum monostearate, glycerin monostearate, cyclodextrin, dextran, and others.

Chelating agents: succinic acid, trihydroxymethylaminomethane, nitrilotriacetic acid, 1-hydroxyethane1-,1-diphosphonic acid, polyphosphoric acid, metaphosphoric acid, hexametaphosphoric acid, and others.

Flavoring agents, or cooling and/or refreshing agents: menthol, camphor, borneol, geraniol, cineol, citronellol, carvone, anethole, eugenol, cineole, limonene, linalyl acetate, borneol, menthone, and others. These may be in any of a d-form, an l-form, and a dl-form, and may be blended as a refined oil (peppermint oil, cool mint oil, spearmint oil, peppermint oil, fennel oil, cinnamon oil, rose oil, or the like).

Preservatives excluding chlorhexidine, sorbic acid, and salts thereof: dibutylhydroxytoluene, butylhydroxyanisole, alkyldiaminoethylglycine hydrochloride, sodium benzoate, ethanol, tyloxapol, benzalkonium chloride, benzethonium chloride, zinc chloride, chlorobutanol, sodium dehydroacetate, methyl paraoxybenzoate, ethyl paraoxybenzoate, propyl paraoxybenzoate, butyl paraoxybenzoate, oxyquinoline sulfate, phenethyl alcohol, benzyl alcohol, biguanide compounds (specifically, polyhexanide hydrochloride (polyhexamethylenebiguanide) and the like), polidronium chloride, chlorocresol, parachlorometaxylenol, Glokill (trade name, manufactured by Rhodia S.A.), and others.

It is sufficient that the water used in the aqueous ophthalmic composition of the present invention is a physiologically or pharmaceutically acceptable one. Examples of such water include distilled water, ordinary water, purified water, sterilized purified water, water for injection, and distilled water for injection. The definition of these is based on The Japanese Pharmacopoeia Sixteenth Edition.

In the present invention, the “salt” may be, for example, a basic salt such as a salt with an inorganic base such as an alkali metal salt or an alkaline earth metal salt, or a salt with an organic base, and examples thereof include salts with sodium, potassium, calcium, magnesium, ammonium, diethanolamine, or ethylenediamine. These salts can be obtained, for example, by converting a sulfuric acid group or a carboxyl group that is present in liranaftate or the like into a salt by a known method. Furthermore, examples of the salts include salts of amine such as ammonia, methylamine, dimethylamine, trimethylamine, dicyclohexylamine, tris(hydroxymethyl)aminomethane, N,N-bis(hydroxyethyl)piperazine, 2-amino-2-methyl-1-propanol, ethanolamine, N-methylglucamine, and L-glucamine; and salts with a basic amino acid such as lysine, δ-hydroxylysine, and arginine. Also, in the present invention, the “salt” may be an acidic salt or the like, and examples thereof include salts with an inorganic acid such as salts with a mineral acid such as hydrochloric acid, hydrobromic acid, sulfuric acid, nitric acid, or phosphoric acid; salts with an organic acid such as methanesulfonic acid, benzenesulfonic acid, paratoluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid, salicylic acid, gluconic acid, or palmitic acid; and salts with an acidic amino acid such as aspartic acid or glutamic acid.

The “physiologically or pharmaceutically acceptable salts” in the present invention may include solvates or hydrates of salts.

It is sufficient that the aqueous ophthalmic composition of the present invention is in a form containing water, and may be, for example, in any of an aqueous solution form, a gel form, a suspension form, and an emulsion form, and is preferably in an aqueous solution form.

The aqueous ophthalmic composition of the present invention may preferably have the following composition, although not limited. Aqueous ophthalmic composition containing sodium hyaluronate, boric acid, and water; aqueous ophthalmic composition containing sodium hyaluronate, epsilon-aminocaproic acid, and water; aqueous ophthalmic composition containing sodium hyaluronate, sodium hydrogen phosphate, and water; aqueous ophthalmic composition containing sodium hyaluronate, sodium citrate, and water; aqueous ophthalmic composition containing sodium hyaluronate, sodium hydrogen carbonate, and water; aqueous ophthalmic composition containing sodium hyaluronate, 2-amino-2-hydroxymethyl-1,3-propanediol, and water; aqueous ophthalmic composition containing sodium chondroitin sulfate, boric acid, and water; aqueous ophthalmic composition containing sodium chondroitin sulfate, epsilon-aminocaproic acid, and water; aqueous ophthalmic composition containing sodium chondroitin sulfate, sodium hydrogen phosphate, and water; aqueous ophthalmic composition containing sodium chondroitin sulfate, sodium citrate, and water; aqueous ophthalmic composition containing sodium chondroitin sulfate, sodium hydrogen carbonate, and water; aqueous ophthalmic composition containing sodium chondroitin sulfate, 2-amino-2-hydroxymethyl-1,3-propanediol, and water; aqueous ophthalmic composition containing glucose, boric acid, and water; aqueous ophthalmic composition containing glucose, epsilon-aminocaproic acid, and water; aqueous ophthalmic composition containing glucose, sodium hydrogen phosphate, and water; aqueous ophthalmic composition containing glucose, sodium citrate, and water; aqueous ophthalmic composition containing glucose, sodium hydrogen carbonate, and water; aqueous ophthalmic composition containing glucose, 2-amino-2-hydroxymethyl-1,3-propanediol, and water.

The pH of the aqueous ophthalmic composition of the present invention is not limited as long as the pH is within a physiologically or pharmaceutically acceptable range; however, the pH may be, for example, 3 or more, preferably 4 or more, more preferably 5 or more, further preferably 5.5 or more, and still more preferably 6 or more. The pH may be, for example, 9 or less, preferably 8.5 or less, more preferably 8 or less, further preferably 7.5 or less, and still more preferably 7 or less.

The osmotic pressure ratio of the aqueous ophthalmic composition of the present invention is suitably set in accordance with the kinds and the content of the blended components, and the purpose of use, the form of the formulation, the method of using, and the like of the aqueous ophthalmic composition as long as the osmotic pressure ratio is within a physiologically or pharmaceutically acceptable range; however, the osmotic pressure ratio can be set to be, for example, from 0.4 to 5, preferably from 0.5 to 4, more preferably from 0.6 to 3, and still more preferably from 0.7 to 2. Here, in the aqueous ophthalmic composition of the present invention, the osmotic pressure ratio is determined as an osmotic pressure ratio relative to a physiological saline solution on the basis of the osmotic pressure measurement method (osmole concentration measurement method) of The Japanese Pharmacopoeia Sixteenth Edition.

The viscosity of the aqueous ophthalmic composition of the present invention is suitably set in accordance with the kinds and the content of the blended components, and the purpose of use, the form of the formulation, the method of using, and the like of the aqueous ophthalmic composition as long as the viscosity is within a physiologically or pharmaceutically acceptable range. The viscosity at 20° C. as measured by using a rotational viscometer (RE550 type viscometer, manufactured by Higashi Sangyo Co., Ltd., rotor; 1° 34′× R24) is preferably set to be from 0.01 to 10000 mPa·s, more preferably from 0.05 to 8000 mPa·s.

The method of using the aqueous ophthalmic composition of the present invention is suitably set in accordance with the kinds and the content of the blended components, and the purpose of use and the form of the formulation of the aqueous ophthalmic composition.

The aqueous ophthalmic composition of the present invention can also prevent deterioration of the PBT-containing resin container, so that the aqueous composition may be used as a deterioration inhibiting agent for a PBT-containing resin container.

Here, although not limited, the prevention of deterioration of the PBT-containing resin container refers to a state with little change in the property of the container when the aqueous ophthalmic composition is used or kept for a predetermined period of time after being stored in the PBT-containing resin container. For example, the prevention of deterioration refers to a state with little change in weight of the container. From the viewpoint of inhibiting deterioration, the change in weight is preferably restrained as much as possible. When an aqueous ophthalmic composition is stored in a PBT-containing resin container, change in weight of the PBT-containing resin occurs, resulting in problems such as decrease in the strength of the container, cracks, deformation, and decrease in the sealing property. That is, when a container formed of a PBT-containing resin is used in a mode in which a pharmaceutical agent such as an aqueous ophthalmic composition is used while being stored for a predetermined period of time, the problem of change in the property of the PBT-containing resin container becomes serious.

The aqueous ophthalmic composition of the present invention can also improve the liquid-cutting property of the PBT-containing resin container and can prevent the liquid from remaining in the container, so that the aqueous composition may be used as a liquid-cut improving agent for a PBT-containing resin container.

Here, although not limited, the liquid-cutting improving agent for a PBT-containing resin container includes a case meaning that the container is less likely to be wetted with the aqueous ophthalmic composition. An index showing the unlikeliness of the wetting of the container with the aqueous ophthalmic composition in this manner can be expressed, for example, by a magnitude of an advancing contact angle which is a dynamic contact angle. The larger the advancing contact angle is, the less likely the container is wetted, showing a state of good liquid-cutting. The smaller the advancing contact angle is or the larger the absolute value of the negative value is, the more likely the container is wetted, showing a poor liquid-cutting.

The aqueous ophthalmic composition of the present invention is provided singly or in a form of a kit by being stored within a PBT-containing resin container. The PBT-containing resin container can be retained in a good manner by storing the aqueous ophthalmic composition of the present invention in the inside and, as a result, the property of the aqueous ophthalmic composition is retained in a good manner after being stored for a long period of time.

The aqueous ophthalmic composition of the present invention is prepared by adding the above (A) component and (B) component, as well as other constituent components as needed to a carrier so as to attain a desired content by using a known preparation method. For example, the aqueous ophthalmic composition of the present invention can be prepared by using a method described in the General Rules of The Japanese Pharmacopoeia Sixteenth Edition. Specifically, for example, the aqueous ophthalmic composition can be prepared by dissolving or suspending the above components in purified water, adjusting the resultant to a predetermined pH and osmotic pressure, and performing a sterilizing treatment with use of a known sterilizing method.

### EXAMPLES

Next, the present invention will be specifically described by way of Examples; however, the present invention is not limited to the following Examples.

<Evaluation 1 of Change in Weight>

### Example 1

Into purified water, sodium hyaluronate (having a weight-average molecular weight of 850,000 to 1,600,000) as the (A) component and boric acid and borax as the (B) component were dissolved at about 70° C., and an aqueous ophthalmic composition was prepared so as to attain the respective concentrations shown in Table 2. The pH thereof was measured at room temperature with use of a HORIBA pH meter.

### Examples 2 to 3

Aqueous ophthalmic compositions of Examples 2 and 3 shown in Table 2 were prepared in the same manner as in Example 1.

### Comparative Examples 1 to 3

Aqueous ophthalmic compositions of Comparative Examples 1 to 3 shown in Table 2 were prepared in the same manner as in Example 1.

TABLE 2

| | Comparative | Comparative | Comparative | | | |
|---|---|---|---|---|---|---|
| Unit: w/v % | Example 1 | Example 2 | Example 3 | Example 1 | Example 2 | Example 3 |
| (A) Sodium hyaluronate | — | 0.1 | — | 0.1 | — | — |
| (A) Glucose | — | — | 0.2 | — | 0.2 | — |
| (A) Sodium chondroitin | — | — | — | — | — | 1 |
| sulfate | | | | | | |
| (B) Boric acid | — | — | — | 0.5 | 0.5 | 0.5 |
| (B) Borax | — | — | — | 0.02 | 0.02 | 0.02 |
| Purified water | Balance | Balance | Balance | Balance | Balance | Balance |
| Total | 100 | 100 | 100 | 1.00 | 100 | 100 |
| pH | 6.8 | 6.1 | 7.1 | 7.0 | 7.0 | 7.0 |
| Weight change per unit | 7.006 | 7.389 | 7.102 | 6.019 | 6.338 | 6.083 |
| volume (mg/cm³) | | | | | | |

(Testing Method)

Transparent glass vials (septum caps) having a capacity of 10 mL were each loaded with 3 mL of respective test liquids of Examples 1 to 3 and Comparative Examples 1 to 3, and further, one PBT-containing resin (product name: PBT NATURAL, manufactured by ARAM Corporation) piece having a diameter of about 1.0 cm, a weight of about 205 mg, and a thickness of about 2.0 mm was immersed into each of the test liquids, followed by quick sealing. After a heat treatment of leaving the glass vials to stand quietly at 70° C. for 2 weeks in a thermostat, the weight of each piece of resin piece was measured, so as to calculate the change in weight per unit volume by Formula 1. This heat treatment corresponds to a case of storage at room temperature for about 3 years. Evaluation can also be made by change in weight relative to the initial weight. The volume of the PBT-containing resin can also be calculated from the density and weight of the resin.

Change in weight per unit volume (mg/cm³)=(weight of resin piece after heat treatment−weight of resin piece before heat treatment)/resin volume (Formula 1)

The result of the test conducted in this manner is as shown in the bottom part of Table 2.

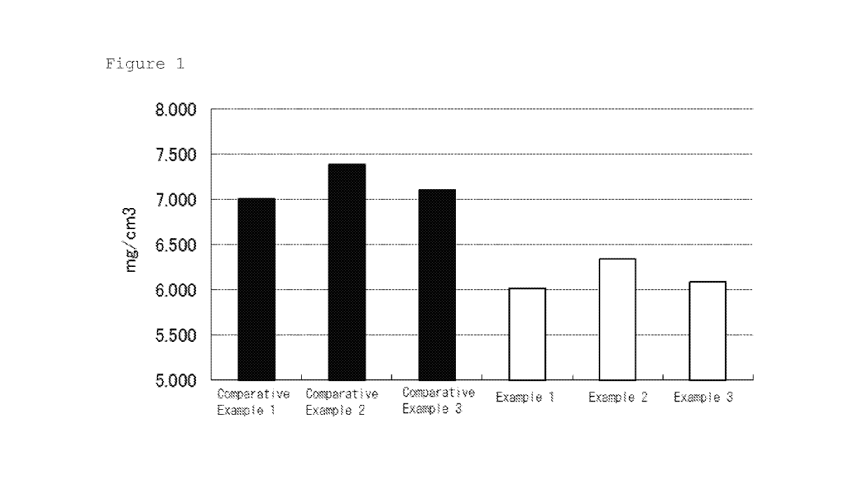

Further, this result was converted into a graph as shown in FIG. 1.

As shown in Table 2 and FIG. 1, in Comparative Example 1 in which the PBT-containing resin piece is immersed into purified water, increase in weight of the PBT-containing resin piece is recognized as compared with the weight before the heat treatment. In contrast, it has been confirmed that, in Comparative Examples 2 and 3 containing sodium hyaluronate which is an acidic polysaccharide or glucose which is a monosaccharide, increase in weight of the PBT-containing resin piece is larger that of the case of purified water. However, in Examples 1 to 3 in which boric acid and borax have been added, the change in weight per unit volume of the PBT resin has been inhibited to be smaller as compared with the case of purified water alone (Comparative Example 1). From this, it has been confirmed that the change in weight of the PBT-containing resin caused by the aqueous ophthalmic composition containing at least one kind selected from the group consisting of an acidic polysaccharide and a monosaccharide is inhibited by boric acid and borax, and deterioration of the PBT-containing resin accompanying this is inhibited. The action mechanism is not clear; however, there seemed to be a possibility that, by the buffering function of the (B) component, the hydrogen ions of the aqueous ophthalmic composition give a certain influence on the chain molecular structure of the PBT-containing resin.

<Evaluation 2 of Change in Weight>

### Examples 4 to 10

Aqueous ophthalmic compositions shown in Table 3 to Table 6 were prepared. The pH thereof was measured at room temperature with use of a HORIBA pH meter.

### Comparative Examples 4 to 12

Aqueous ophthalmic compositions of Comparative Examples 4 to 12 shown in Table 3 to Table 6 were prepared in the same manner as in Examples 4 to 10.

TABLE 3

| | Comparative | Comparative | | Comparative | |
|---|---|---|---|---|---|
| Unit: w/v % | Example 4 | Example 5 | Example 4 | Example 6 | Example 5 |
| (A) Retinol palmitate | — | 50,000 units | 50,000 units | — | — |
| (B) Boric acid | — | — | 0.5 | — | 0.5 |
| (B) Borax | — | — | 0.02 | — | 0.02 |
| (A) Polyoxyethylene | — | 0.2 | 0.2 | 0.2 | 0.2 |
| hydrogenated castor oil 60 | | | | | |
| Tocopherol acetate | — | — | — | 0.05 | 0.05 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.0 | 7.0 | 7.0 | 7.0 | 7.1 |
| Weight change per unit volume | 7.692 | 9.554 | 1.274 | 9.554 | 2.229 |
| (mg/cm³) | | | | | |

TABLE 4

| | Comparative | Comparative | |
|---|---|---|---|
| Unit: w/v % | Example 7 | Example 8 | Example 6 |
| (A) Retinol palmitate | — | 50,000 units | 50,000 units |
| (B) Boric acid | — | — | 0.5 |
| (B) Borax | — | — | 0.02 |
| (A) Polyoxyethylene hydrogenated | — | 0.2 | 0.2 |
| castor oil 60 | | | |
| Hydrochloric acid | Suitable amount | Suitable amount | Suitable amount |
| Sodium hydroxide | Suitable amount | Suitable amount | Suitable amount |
| Purified water | Balance | Balance | Balance |
| Total | 100 | 100 | 1.00 |
| pH | 5.0 | 5.1 | 5.2 |
| Weight change per unit volume (mg/cm³) | 7.643 | 7.962 | 0.000 |

TABLE 5

| | | | | Comparative | | Comparative | |
|---|---|---|---|---|---|---|---|
| | Comparative | Comparative | | Example | | Example | |
| Unit: w/v % | Example 4 | Example 9 | Example 7 | 10 | Example 8 | 11 | Example 9 |
| (A) Sesame oil | — | 0.02 | 0.02 | — | — | — | — |
| (A) Polyoxyethylene castor oil | — | — | — | 0.04 | 0.04 | — | — |
| 35 | | | | | | | |
| (A) Vaseline | — | — | — | — | — | 0.001 | 0.001 |
| (B) Boric acid | — | — | 0.5 | — | 0.5 | — | 0.5 |
| (B) Borax | — | — | 0.02 | — | 0.02 | — | 0.02 |
| (A) Polyoxyethylene | — | 0.2 | 0.2 | 0.2 | 0.2 | 0.2 | 0.2 |
| hydrogenated castor oil 60 | | | | | | | |
| Hydrochloric acid | Suitable | Suitable | Suitable | Suitable | Suitable | Suitable | Suitable |
| | amount | amount | amount | amount | amount | amount | amount |
| Sodium hydroxide | Suitable | Suitable | Suitable | Suitable | Suitable | Suitable | Suitable |
| | amount | amount | amount | amount | amount | amount | amount |
| Purified water | Balance | Balance | Balance | Balance | Balance | Balance | Balance |
| Total | 100 | 100 | 100 | 100 | 100 | 100 | 100 |
| pH | 7.0 | 7.0 | 7.0 | 7.0 | 7.0 | 7.0 | 7.0 |
| Weight change per unit volume | 7.962 | 11.465 | 1.274 | 10.510 | 0.000 | 8.917 | 0.318 |
| (mg/cm³) | | | | | | | |

TABLE 6

| | Comparative | Comparative | |
|---|---|---|---|
| Unit: w/v % | Example 7 | Example 12 | Example 10 |
| (A) Cyanocobalamine | — | 0.02 | 0.02 |
| (B) Boric acid | — | — | 0.5 |
| (B) Borax | — | — | 0.02 |
| Hydrochloric acid | Suitable amount | Suitable amount | Suitable amount |
| Sodium hydroxide | Suitable amount | Suitable amount | Suitable amount |
| Purified water | Balance | Balance | Balance |
| Total | 100 | 100 | 100 |
| pH | 5.0 | 5.2 | 5.2 |
| Weight change per unit volume (mg/cm³) | 7.643 | 9.873 | 7.325 |

(Testing Method)

Transparent glass vials (septum caps) having a capacity of 10 mL were each loaded with 3 mL of respective test liquids of Examples 4 to 10 and Comparative Examples 4 to 12, and further, one PBT-containing resin (product name: PBT NATURAL, manufactured by ARAM Corporation) piece having a diameter of about 1.0 cm, a weight of about 205 mg, and a thickness of about 2.0 mm was immersed into each of the test liquids, followed by quick sealing. After a heat treatment of leaving the glass vials to stand quietly at 75° C. for 2 weeks in a thermostat, the weight of each piece of resin was measured, so as to calculate the change in weight per unit volume by Formula 1. Evaluation can also be made by change in weight relative to the initial weight. The volume of the PBT-containing resin can also be calculated from the density and weight of the resin.

Change in weight per unit volume (mg/cm³)=(weight of resin piece after heat treatment−weight of resin piece before heat treatment)/resin volume (Formula 1)

The result of the test conducted in this manner is as shown in the bottom part of Tables 3 to 6.

As shown in the Tables, in each of the Comparative Examples, increase in weight of the PBT-containing resin piece was recognized as compared with the weight before the heat treatment. However, in the Examples in which boric acid and borax had been added, the change in weight per unit volume of the PBT-containing resin was suppressed to be smaller than that of Comparative Examples. From this, it has been confirmed that the change in weight of the PBT-containing resin caused by the aqueous ophthalmic composition containing the (A) components is suppressed by boric acid and borax, and deterioration of the PBT-containing resin accompanying this is inhibited. The action mechanism is not clear; however, there seemed to be a possibility that, by the buffering function of the (B) component, the hydrogen ions of the aqueous ophthalmic composition give a certain influence on the chain molecular structure of the PBT-containing resin.

<Evaluation 3 of Change in Weight>

### Examples 11 to 18

Aqueous ophthalmic compositions were prepared so as to attain a concentration shown in Table 7 to Table 13. The pH thereof was measured at room temperature with use of a HORIBA pH meter.

### Comparative Examples 13 to 22

Aqueous ophthalmic compositions of Comparative Examples 13 to 22 shown in Table 7 to Table 13 were prepared in the same manner as in Examples 11 to 18.

TABLE 7

| | Comparative | Comparative | Example | Comparative | Example |
|---|---|---|---|---|---|
| Unit: w/v % | Example 13 | Example 14 | 11 | Example 15 | 12 |
| (A) Retinol palmitate | — | 30,000 | 30,000 | — | — |
| | | units | units | | |
| (B) Boric acid | — | — | 1.8 | — | 1 |
| (B) Borax | — | — | 0.1 | — | — |
| (B) Sodium hydrogen phosphate | — | — | — | — | 0.5 |
| (B) Epsilon-aminocaproic acid | — | — | 1 | — | — |
| (A) Polyoxyethylene | — | — | — | 0.2 | 0.2 |
| hydrogenated castor oil 60 | | | | | |
| Tocopherol acetate | — | — | — | 0.01 | 0.01 |
| Polysorbate 80 | — | 0.1 | 0.1 | — | — |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 4.6 | 4.5 | 4.5 | 5.0 | 5.0 |
| Weight change per unit volume | 7.325 | 8.917 | 5.096 | 8.280 | 4.459 |
| (mg/cm³) | | | | | |

TABLE 8

| | Comparative | Comparative | |
|---|---|---|---|
| Unit: w/v % | Example 16 | Example 17 | Example 13 |
| (A) Cyanocobalamine | — | 0.004 | 0.004 |
| (B) Sodium citrate | — | — | 0.1 |
| (B) Epsilon-aminocaproic acid | — | — | 3 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 7.0 | 7.0 |
| Weight change per unit | 7.643 | 12.102 | 4.459 |
| volume (mg/cm³) | | | |

TABLE 9

| | Comparative | Comparative | |
|---|---|---|---|
| Unit: w/v % | Example 13 | Example 18 | Example 14 |
| (A) Sesame oil | — | 0.1 | 0.1 |
| (B) Boric acid | — | — | 1.2 |
| (B) Borax | — | — | 0.05 |
| (B) | — | — | 5 |
| 2-amino-2-hydroxymethyl- | | | |
| 1,3-propanediol | | | |
| (A) Polyoxyethylene | — | 1 | 1 |
| hydrogenated castor oil 60 | | | |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 4.6 | 5.0 | 5.0 |
| Weight change per unit | 7.325 | 10.191 | 7.006 |
| volume (mg/cm³) | | | |

TABLE 10

| | Comparative | Comparative | Example |
|---|---|---|---|
| Unit: w/v % | Example 16 | Example 19 | 15 |
| (A) Castor oil | — | 0.3 | 0.3 |
| (B) Sodium hydrogen phosphate | — | — | 1.5 |
| (B) Sodium citrate | — | — | 0.5 |
| (A) Polyoxyethylene | — | 1 | 1 |
| hydrogenated castor oil 60 | | | |
| Polysorbate 80 | — | 1.5 | 1.5 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 7.0 | 7.0 |
| Weight change per unit volume | 7.643 | 8.280 | 7.006 |
| (mg/cm³) | | | |

TABLE 11

| | Comparative | Comparative | Example |
|---|---|---|---|
| Unit: w/v % | Example 13 | Example 20 | 16 |
| (A) Polyoxyethylene castor | — | 0.4 | 0.4 |
| oil 10 | | | |
| (B) Boric acid | — | — | 1.8 |
| (B) Sodium citrate | — | — | 0.3 |
| (A) Polyoxyethylene | — | 2 | 2 |
| hydrogenated castor oil 60 | | | |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 4.6 | 5.0 | 5.0 |
| Weight change per unit volume | 7.325 | 8.917 | 5.096 |
| (mg/cm³) | | | |

TABLE 12

| | Comparative | Comparative | Example |
|---|---|---|---|
| Unit: w/v % | Example 16 | Example 21 | 17 |
| (A) Liquid paraffin | — | 0.02 | 0.02 |
| (B) Sodium hydrogen phosphate | — | — | 1 |
| (B) Sodium dihydrogen | — | — | 0.2 |
| phosphate | | | |
| (B) Sodium citrate | — | — | 0.5 |
| (A) Polyoxyethylene | — | 2 | 2 |
| hydrogenated castor oil 60 | | | |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 7.0 | 7.0 |
| Weight change per unit volume | 7.643 | 9.554 | 7.643 |
| (mg/cm³) | | | |

TABLE 13

| | Comparative | Comparative | Example |
|---|---|---|---|
| Unit: w/v % | Example 13 | Example 22 | 18 |
| (A) Vaseline | — | 0.01 | 0.01 |
| (B) Boric acid | — | — | 1.5 |
| (B) Borax | — | — | 0.3 |
| (B) | — | — | 1 |
| 2-amino-2-hydroxymethyl- | | | |
| 1,3-propanediol | | | |
| (A) Polyoxyethylene | — | 2 | 2 |
| hydrogenated castor oil 60 | | | |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 4.6 | 5.0 | 5.0 |
| Weight change per unit volume | 7.325 | 9.554 | 6.369 |
| (mg/cm³) | | | |

(Testing Method)

Transparent glass vials (septum caps) having a capacity of 10 mL were each loaded with 2 mL of respective test liquids of Examples and Comparative Examples, and further, one PBT-containing resin (product name: PBT NATURAL, manufactured by ARAM Corporation) piece having a diameter of about 1.0 cm, a weight of about 205 mg, and a thickness of about 2.0 mm was immersed into each of the test liquids, followed by quick sealing. After a heat treatment of leaving the glass vials to stand quietly at 75° C. for one week in a thermostat, the weight of each piece of resin was measured, so as to calculate the change in weight per unit volume by Formula 1. Evaluation can also be made by change in weight relative to the initial weight. The volume of the PBT-containing resin can also be calculated from the density and weight of the resin.

Change in weight per unit volume (mg/cm³)=(weight of resin piece after heat treatment−weight of resin piece before heat treatment)/resin volume (Formula 1)

The result of the test conducted in this manner is as shown in the bottom part of Tables 7 to 13.

As shown in the Tables, in each of the Comparative Examples, increase in weight of the PBT-containing resin piece is recognized as compared with the weight before the heat treatment. However, in the Examples in which the buffering agent had been added, change in weight per unit volume of the PBT-containing resin was suppressed to be smaller than that of Comparative Examples. From this, it has been confirmed that the change in weight of the PBT-containing resin caused by the aqueous ophthalmic composition containing the (A) components is suppressed by the buffering agent, and deterioration of the PBT-containing resin accompanying this is inhibited. The action mechanism is not clear; however, there seemed to be a possibility that, by the buffering function of the (B) component, the hydrogen ions of the aqueous ophthalmic composition give a certain influence on the chain molecular structure of the PBT-containing resin.

<Evaluation 1 of Advancing Contact Angle>

### Examples 19 to 87

Aqueous ophthalmic compositions shown in Tables 14 to 32 were prepared by an ordinary method and used as test liquids. With use of a contact angle meter DM-501 manufactured by Kyowa Interface Science Co., Ltd., an advancing contact angle, which is a contact angle when an interface between a solid and a liquid moves, was measured in accordance with the measurement procedure of the extension/contraction method of the measurement apparatus. Specifically, a plate-shaped PBT-containing resin (product name: PBT NATURAL, manufactured by ARAM Corporation), which is a square prism having one side being 50 mm in length and a thickness of about 2 mm, was placed on a stage of the contact angle meter, and the test liquid was set in the dispenser. A liquid droplet having a volume of 1 μL of the test liquid was dropped onto the PBT-containing resin plate so as to be attached thereto in a hemispherical shape. Subsequently, the tip end of a liquid ejection part of the dispenser was quickly brought into contact with the upper part of the hemisphere. In that state, the test liquid was continuously ejected at an ejection speed of 6 μL/second, and images of the shape of the liquid droplet were captured from the side surface for 15 times per 0.1 second. In order to match the measurement conditions of the corresponding Comparative Examples with those of the Examples, the measurement was continuously carried out at the same room temperature, and the same plate-shaped PBT-containing resin was used. Subsequently, with use of an analyzing software FAMAS of the measurement apparatus, the contact angles on the right and on the left were determined for each image. Here, the contact angle means an angle which is one of the two angles formed by a tangential line drawn from the contact point P of the surface of the PBT-containing resin plate, the test liquid, and the air to the test liquid and a tangential line drawn to the surface of the PBT-containing resin plate and which is on the side including the test liquid. A behavior was seen such that, as the liquid droplet extended by ejection of the test liquid, the contact angle changed and then became approximately constant. Then, an average value of the contact angles on the right and on the left was calculated for each image. The average values of the right and left contact angles were listed sequentially in the order in which the images had been captured. When five consecutive average values of the right and left contact angles listed above were selected, the first contact angle at which the standard deviation of the five consecutive average values of the right and left contact angles became 2.5° or less for the first time was determined as the advancing contact angle of the present invention. This was carried out for three times for each test liquid so as to determine the advancing contact angles, and an average value of the advancing contact angles of the three times was determined as an advancing contact angle of the test liquid. Also in the case in which the advancing contact angle did not change during the process in which the liquid droplet extended, the first contact angle at which the standard deviation of the five consecutive average values of the right and left contact angles became 2.5° or less for the first time was similarly determined as the advancing contact angle of the present invention. By the following Formula (2), the rise rate of the advancing contact angle of the Example relative to the advancing contact angle of the corresponding Comparative Example was calculated.

Rise rate (%)={(advancing contact angle of each test liquid/advancing contact angle of Comparative Example)−1}×100 <Formula (2)>

The Comparative Examples refer to aqueous ophthalmic compositions from which the (B) components contained in the Examples have been removed. For example, the Comparative Example corresponding to Example 19 of Table 14 is an aqueous ophthalmic composition which contains 0.5 w/v % of sodium chondroitin sulfate and which is adjusted with use of hydrochloric acid or sodium hydroxide to have a pH value of 5.1 that is equal to the pH of Example 19.

Also, unless specifically noted in the Tables here, the test liquid was subjected to the test immediately after being prepared.

TABLE 14

| | Example | Example | Example | Example | Example | Example | Example | Example |
|---|---|---|---|---|---|---|---|---|
| Unit: w/v % | 19 | 20 | 21 | 22 | 23 | 24 | 25 | 26 |
| (A) Sodium chondroitin sulfate | 0.5 | 0.5 | 0.5 | 0.5 | 1 | 0.05 | 0.5 | 0.25 |
| (B) Boric acid | 0.5 | 0.5 | — | — | — | 0.5 | 2.0 | — |
| (B) Borax | 0.02 | 0.02 | — | — | — | 0.02 | 0.1 | — |
| (B) Sodium hydrogen phosphate | — | — | — | — | 0.5 | — | — | — |
| (B) Sodium dihydrogen phosphate | — | — | — | — | 0.02 | — | — | — |
| (B) Sodium citrate | — | — | 1 | 0.1 | — | — | — | — |
| (B) Epsilon-aminocaproic acid | — | — | — | — | — | — | — | 2 |
| Hydrochloric acid | Suitable | Suitable | Suitable | Suitable | Suitable | Suitable | Suitable | Suitable |
| | amount | amount | amount | amount | amount | amount | amount | amount |
| Sodium hydroxide | Suitable | Suitable | Suitable | Suitable | Suitable | Suitable | Suitable | Suitable |
| | amount | amount | amount | amount | amount | amount | amount | amount |
| Purified water | Balance | Balance | Balance | Balance | Balance | Balance | Balance | Balance |
| Total | 100 | 100 | 100 | 100 | 100 | 100 | 100 | 100 |
| pH | 5.1 | 7.9 | 7.9 | 4.9 | 7.8 | 7.0 | 7.0 | 7.0 |
| Rise rate | 8.7% | 11.7% | 16.9% | 11.6% | 20.8% | 19.4% | 10.9% | 30.9% |

TABLE 15

| Unit: w/v % | Example 27 | Example 28 | Example 29 |
|---|---|---|---|
| (A) Cyanocobalamine | 0.02 | 0.02 | 0.004 |
| (B) Boric acid | 0.5 | 0.5 | — |
| (B) Borax | 0.02 | 0.02 | — |
| (B) Sodium citrate | — | — | 0.1 |
| (B) Epsilon-aminocaproic acid | — | — | 3 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 6.9 | 5.2 | 7.0 |
| Rise rate | 12.4% | 10.4% | 10.1% |

TABLE 16

| Unit: w/v % | Example 30 | Example 31 | Example 32 | Example 33 | Example 34 |
|---|---|---|---|---|---|
| (A) Polyoxyethylene castor oil 10 | 0.04 | 0.4 | — | — | — |
| (A) Poloxamer 407 | — | — | 0.1 | 0.1 | 2 |
| (B) Boric acid | 0.5 | 1.8 | 1 | 1 | — |
| (B) Borax | 0.02 | — | 0.04 | 0.04 | — |
| (B) Sodium hydrogen phosphate | — | — | 1 | — | 0.8 |
| (B) Sodium dihydrogen | — | — | 0.2 | — | 0.1 |
| phosphate | | | | | |
| (B) Sodium citrate | — | 0.3 | — | — | — |
| (A) Polyoxyethylene | 0.2 | 2 | 0.1 | — | — |
| hydrogenated castor oil 60 | | | | | |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.0 | 5.0 | 7.5 | 7.6 | 5.1 |
| Rise rate | 21.6% | 18.5% | 27.1% | 10.4% | 7.2% |

TABLE 17

| | Example | Example | Example | Example | Example | Example |
|---|---|---|---|---|---|---|
| Unit: w/v % | 35 | 36 | 37 | 38 | 39 | 40 |
| (A) Retinol palmitate | 50,000 | 50,000 | 50,000 | 40,000 | 20,000 | — |
| | units | units | units | units | units | |
| (B) Boric acid | 0.5 | 0.5 | 0.5 | — | — | 0.5 |
| (B) Borax | 0.02 | 0.02 | 0.02 | — | — | 0.02 |
| (B) Epsilon-aminocaproic acid | — | — | — | — | 2.5 | — |
| (B) | — | — | — | 1 | — | — |
| 2-amino-2-hydroxymethyl-1,3-propanediol | | | | | | |
| (A) Polyoxyethylene | 0.2 | 0.2 | — | 0.13 | 0.3 | 0.2 |
| hydrogenated castor oil 60 | | | | | | |
| Tocopherol acetate | — | — | — | — | — | 0.05 |
| Polysorbate 80 | — | — | 0.2 | — | — | — |
| Hydrochloric acid | Suitable | Suitable | Suitable | Suitable | Suitable | Suitable |
| | amount | amount | amount | amount | amount | amount |
| Sodium hydroxide | Suitable | Suitable | Suitable | Suitable | Suitable | Suitable |
| | amount | amount | amount | amount | amount | amount |
| Purified water | Balance | Balance | Balance | Balance | Balance | Balance |
| Total | 100 | 100 | 100 | 100 | 100 | 100 |
| pH | 7.0 | 5.2 | 7.0 | 7.1 | 4.8 | 7.0 |
| Rise rate | 8.6% | 5.7% | 10.3% | 7.1% | 16.7% | 5.3% |

TABLE 18

| | Example | Example | Example | Example | Example |
|---|---|---|---|---|---|
| Unit: w/v % | 41 | 42 | 43 | 44 | 45 |
| (A) Retinol palmitate | 10,000 | 30,000 | 30,000 | — | — |
| | units | units | units | | |
| (B) Boric acid | 0.5 | 0.2 | 1.8 | 1 | — |
| (B) Borax | 0.02 | 0.01 | 0.1 | — | — |
| (B) Sodium hydrogen phosphate | — | 0.7 | — | 0.5 | — |
| (B) Sodium citrate | — | 0.5 | — | — | — |
| (B) Epsilon-aminocaproic acid | — | — | 1 | — | 2 |
| (B) 2-amino-2-hydroxymethyl-1,3-propanediol | 1 | — | — | — | — |
| (A) Polyoxyethylene hydrogenated castor | 0.1 | 0.4 | — | 0.2 | 0.2 |
| oil 60 | | | | | |
| Tocopherol acetate | — | — | — | 0.01 | 0.01 |
| Polysorbate 80 | — | — | 0.1 | — | — |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.5 | 7.0 | 4.5 | 5.0 | 5.0 |
| Rise rate | 23.5% | 9.9% | 6.3% | 17.7% | 17.5% |

In Examples 41 to 45 and corresponding Comparative Examples, a liquid subjected to a heat treatment at 80° C. for one day was used for the test.

TABLE 19

| | Example | Example | Example |
|---|---|---|---|
| Unit: w/v % | 46 | 47 | 48 |
| (A) Sesame oil | 0.02 | 0.1 | — |
| (A) Castor oil | — | — | 0.3 |
| (B) Boric acid | 0.5 | 1.2 | — |
| (B) Borax | 0.02 | 0.05 | — |
| (B) Sodium hydrogen phosphate | — | — | 1.5 |
| (B) Sodium dihydrogen phosphate | — | — | 0.5 |
| (B) Sodium citrate | — | — | — |
| (B) 2-amino-2-hydroxymethyl- | — | 5 | — |
| 1,3-propanediol | | | |
| (A) Polyoxyethylene | 0.2 | 1 | 1 |
| hydrogenated castor oil 60 | | | |
| Polysorbate 80 | — | — | 1.5 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 5.0 | 7.0 |
| Rise rate | 10.3% | 7.4% | 7.9% |

TABLE 20

| | Example | Example | Example |
|---|---|---|---|
| Unit: w/v % | 49 | 50 | 51 |
| (A) White vaseline | 0.001 | 0.01 | — |
| (A) Liquid paraffin | — | — | 0.02 |
| (B) Boric acid | 0.5 | 1.5 | — |
| (B) Borax | 0.02 | 0.3 | — |
| (B) Sodium hydrogen phosphate | — | — | 1 |
| (B) Sodium dihydrogen phosphate | — | — | 0.2 |
| (B) Sodium citrate | — | — | 0.5 |
| (B) 2-amino-2-hydroxymethyl- | — | 1 | — |
| 1,3-propanediol | | | |
| (A) Polyoxyethylene | 0.2 | 2 | 2 |
| hydrogenated castor oil 60 | | | |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 5.0 | 7.0 |
| Rise rate | 7.0% | 14.1% | 22.5% |

TABLE 21

| Unit: w/v % | Example 52 | Example 53 | Example 54 | Example 55 | Example 56 |
|---|---|---|---|---|---|
| (A) Sodium hyaluronate | 0.005 | — | — | — | — |
| (A) Alginic acid | — | 0.1 | 0.1 | — | — |
| (A) Gellan gum | — | — | — | 0.1 | — |
| (A) Xanthan gum | — | — | — | — | 0.5 |
| (B) Boric acid | 0.5 | — | 0.6 | 0.6 | 0.6 |
| (B) Borax | 0.02 | — | 0.02 | 0.02 | 0.02 |
| (B) Epsilon-aminocaproic acid | — | 1 | — | — | — |
| (B) Sodium hydrogen phosphate | — | 0.5 | — | — | — |
| (B) Sodium dihydrogen | — | 0.1 | — | — | — |
| phosphate | | | | | |
| Edetate sodium | — | — | — | 0.1 | — |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.0 | 7.1 | 7.1 | 7.0 | 7.0 |
| Rise rate | 3.0% | 8.7% | 6.9% | 8.9% | 6.8% |

TABLE 22

| Unit: w/v % | Example 57 |
|---|---|
| (A) Glucose | 0.05 |
| (B) Boric acid | 0.05 |
| (B) Borax | 0.02 |
| Hydrochloric acid | Suitable amount |
| Sodium hydroxide | Suitable amount |
| Purified water | Balance |
| Total | 100 |
| pH | 7.0 |
| Rise rate | 7.9% |

TABLE 23

| Unit: w/v % | Example 58 | Example 59 |
|---|---|---|
| (A) Flavin adenine dinucleotide | 0.05 | — |
| sodium | | |
| (A) Panthenol | — | 0.1 |
| (B) Boric acid | 0.7 | 1.2 |
| (B) Borax | 0.01 | 0.4 |
| Hydrochloric acid | Suitable amount | Suitable amount |
| Sodium hydroxide | Suitable amount | Suitable amount |
| Purified water | Balance | Balance |
| Total | 100 | 100 |
| pH | 4.9 | 4.9 |
| Rise rate | 10.0% | 2.8% |

TABLE 24

| Unit: w/v % | Example 60 | Example 61 |
|---|---|---|
| (A) Castor oil | 0.02 | — |
| (A) Refined lanolin | — | 0.05 |
| (A) Polyoxyethylene | 1.1 | 1 |
| hydrogenated | | |
| castor oil 60 | | |
| (B) Boric acid | 0.6 | 0.6 |
| (B) Borax | 0.02 | 0.02 |
| Hydrochloric acid | Suitable | Suitable |
| | amount | amount |
| Sodium hydroxide | Suitable | Suitable |
| | amount | amount |
| Purified water | Balance | Balance |
| Total | 100 | 100 |
| pH | 7.1 | 7.0 |
| Rise rate | 7.4% | 6.2% |

TABLE 25

| Unit: w/v % | Example 62 | Example 63 | Example 64 | Example 65 | Example 66 |
|---|---|---|---|---|---|
| (A) Polyoxyethylene hydrogenated | 0.4 | 0.4 | | | |
| castor oil 60 | | | | | |
| (A) Polyoxyl 40 stearate | — | — | 0.08 | | — |
| (A) Poloxamer407 | — | — | | 0.5 | 0.5 |
| (B) Boric acid | 1.2 | — | 0.6 | 0.4 | 0.6 |
| (B) Borax | 0.2 | — | 0.02 | 0.015 | 0.02 |
| (B) Sodium hydrogen phosphate | — | 0.8 | — | — | — |
| (B) Sodium dihydrogen phosphate | — | 0.1 | — | — | — |
| (B) Sodium citrate | — | 0.5 | — | — | — |
| (B) 2-amino-2-hydroxymethyl- | — | — | — | — | 0.5 |
| 1,3-propanediol | | | | | |
| (B) Sodium hydrogen carbonate | — | — | — | — | 0.2 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.1 | 7.0 | 7.0 | 7.0 | 7.0 |
| Rise rate | 5.6% | 6.9% | 5.0% | 11.7% | 15.7% |

TABLE 26

| Unit: w/v % | Example 67 | Example 68 | Example 69 | Example 70 |
|---|---|---|---|---|
| (A) Diphenhydramine hydrochloride | 0.05 | — | — | — |
| (A) Ketotifen fumarate | — | 0.069 | — | — |
| (A) Tranilast | — | — | 0.5 | 0.5 |
| (A) Polyvinylpyrrolidone k90 | — | — | 2.5 | — |
| (A) Polyvinylpyrrolidone k25 | — | — | — | 2.5 |
| (B) Boric acid | 0.6 | 0.7 | 1.1 | 0.5 |
| (B) Borax | 0.02 | 0.01 | 0.6 | 0.2 |
| Hydrochloric acid | Suitable | Suitable | Suitable | Suitable |
| | amount | amount | amount | amount |
| Sodium hydroxide | Suitable | Suitable | Suitable | Suitable |
| | amount | amount | amount | amount |
| Purified water | Balance | Balance | Balance | Balance |
| Total | 100 | 100 | 100 | 100 |
| pH | 7.0 | 5.3 | 7.9 | 7.9 |
| Rise rate | 10.5% | 16.7% | 8.7% | 11.8% |

In Example 68 and corresponding Comparative Example, a liquid subjected to a heat treatment at 75° C. for three days was used for the test.

TABLE 27

| Unit: w/v % | Example 71 | Example 72 |
|---|---|---|
| (A) Chlorhexidine | 0.005 | — |
| gluconate | | |
| (A) Potassium sorbate | — | 0.1 |
| (B) Boric acid | 0.6 | 0.6 |
| (B) Borax | 0.02 | 0.02 |
| Hydrochloric acid | Suitable | Suitable |
| | amount | amount |
| Sodium hydroxide | Suitable | Suitable |
| | amount | amount |
| Purified water | Balance | Balance |
| Total | 100 | 100 |
| pH | 7.0 | 7.0 |
| Rise rate | 14.3% | 3.6% |

TABLE 28

| Unit: w/v % | Example 73 | Example 74 | Example 75 | Example 76 | Example 77 |
|---|---|---|---|---|---|
| (A) Polyvinylpyrrolidone k90 | 3.0 | — | — | — | — |
| (A) Polyvinylpyrrolidone k25 | | 2.5 | — | — | — |
| (A) Carboxyvinyl polymer | — | — | 0.3 | — | — |
| (A) Carboxymethylcellulose sodium | — | — | | 0.1 | — |
| (A) Methylcellulose | | | | | 0.4 |
| (B) Boric acid | 0.6 | 1.0 | 0.6 | 1.8 | 0.6 |
| (B) Borax | 0.02 | 0.2 | 0.02 | 0.05 | 0.02 |
| Edetate sodium | | | | 0.08 | — |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.0 | 7.1 | 7.0 | 7.1 | 7.0 |
| Rise rate | 17.7% | 8.4% | 14.9% | 7.8% | 3.0% |

TABLE 29

| Unit: w/v % | Example 78 | Example 79 | Example 80 |
|---|---|---|---|
| (A) Propylene glycol | 1.0 | — | — |
| (A) Glycerin | | 2.0 | — |
| (A) D-mannitol | — | — | 1.0 |
| (B) Boric acid | 0.6 | 0.6 | 0.6 |
| (B) Borax | 0.02 | 0.02 | 0.02 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.0 | 7.0 | 7.0 |
| Rise rate | 21.7% | 7.5% | 14.7% |

TABLE 30

| Unit: w/v % | Example 81 | Example 82 | Example 83 | Example 84 |
|---|---|---|---|---|
| (A) Berberine chloride | 0.025 | — | — | — |
| (A) Allantoin | — | 0.3 | — | — |
| (A) Zinc sulfate | — | — | 0.25 | — |
| (A) Azulene sodium sulfonate | — | — | | 0.02 |
| (B) Boric acid | 0.7 | 0.7 | 0.7 | 0.6 |
| (B) Borax | 0.01 | 0.01 | 0.01 | 0.04 |
| Hydrochloric acid | Suitable | Suitable | Suitable | Suitable |
| | amount | amount | amount | amount |
| Sodium hydroxide | Suitable | Suitable | Suitable | Suitable |
| | amount | amount | amount | amount |
| Purified water | Balance | Balance | Balance | Balance |
| Total | 100 | 100 | 100 | 100 |
| pH | 4.9 | 4.9 | 5.0 | 8.0 |
| Rise rate | 8.3% | 3.3% | 15.4% | 14.1% |

TABLE 31

| Unit: w/v % | Example 85 |
|---|---|
| (A) sulfamethoxazole | 4.0 |
| sodium | |
| (B) Boric acid | 0.6 |
| (B) Borax | 0.04 |
| Hydrochloric acid | Suitable |
| | amount |
| Sodium hydroxide | Suitable |
| | amount |
| Purified water | Balance |
| Total | 100 |
| PH | 8.0 |
| Rise rate | 7.8% |

TABLE 32

| Unit: w/v % | Example 86 | Example 87 |
|---|---|---|
| (A)Eucalyptus oil | 0.005 | — |
| (A)Bergamot oil | — | 0.002 |
| (A) Polyoxyethylene | 0.1 | 0.1 |
| hydrogenated | | |
| castor oil 60 | | |
| (B) Boric acid | 0.6 | 0.6 |
| (B) Borax | 0.02 | 0.02 |
| Hydrochloric acid | Suitable | Suitable |
| | amount | amount |
| Sodium hydroxide | Suitable | Suitable |
| | amount | amount |
| Purified water | Balance | Balance |
| Total | 100 | 100 |
| pH | 7.0 | 7.0 |
| Rise rate | 5.3% | 6.0% |

In Example 87 and corresponding Comparative Example, a liquid subjected to a heat treatment at 75° C. for 3 days was used for the test.

As shown in each Table, when compared with the Comparative Examples that did not contain the (B) components, the rise rate of the Examples was high. From this, it has been confirmed that the aqueous ophthalmic composition obtained by incorporating the (B) components into the aqueous ophthalmic composition containing the (A) components makes the PBT-containing resin less likely be wetted when in movement. It has been found out that an advantageous effect is obtained such as an improvement in liquid-cutting with respect to the PBT-containing resin.

<Evaluation 4 of Change in Weight>

### Examples 88 to 122

Aqueous ophthalmic compositions were prepared so as to contain the (A) components, (B) components, and other components shown in Tables 33 to 53 at concentrations respectively shown in the Tables. The pH thereof was measured at room temperature with use of a HORIBA pH meter.

### Comparative Examples 23 to 63

Aqueous ophthalmic compositions of Comparative Examples shown in Tables 33 to 53 were prepared in the same manner as in the Examples.

(Testing Method)

Transparent glass vials (septum caps) having a capacity of 10 mL were each loaded with 3 mL of respective test liquids of Examples and Comparative Examples, and further, one PBT-containing resin (product name: PBT NATURAL, manufactured by ARAM Corporation) piece having a diameter of about 1.0 cm, a weight of about 205 mg, and a thickness of about 2.0 mm was immersed into each of the test liquids, followed by quick sealing. After a heat treatment of leaving the glass vials to stand quietly in a thermostat at the temperature and for the number of days that are shown in the respective Tables, the weight of each piece of resin was measured, so as to calculate the change in weight per unit volume by Formula 1. Evaluation can also be made by change in weight relative to the initial weight. The volume of the PBT-containing resin can also be calculated from the density and weight of the resin.

Change in weight per unit volume (mg/cm³)=(weight of resin piece after heat treatment−weight of resin piece before heat treatment)/resin volume (Formula 1)

The result of the test conducted in this manner is as shown in the bottom part of the respective Tables.

TABLE 33

| | Comparative | Comparative | | |
|---|---|---|---|---|
| Unit: w/v % | Example 23 | Example 24 | Example 88 | Example 89 |
| (A) Alginic acid | — | 0.1 | 0.1 | 0.1 |
| (B) Boric acid | — | — | — | 0.6 |
| (B) Borax | — | — | — | 0.02 |
| (B) Epsilon-aminocaproic acid | — | — | 1.0 | — |
| (B) Sodium hydrogen phosphate | — | — | 0.5 | — |
| (B) Sodium dihydrogen phosphate | — | — | 0.1 | — |
| Hydrochloric acid | Suitable | Suitable | Suitable | Suitable |
| | amount | amount | amount | amount |
| Sodium hydroxide | Suitable | Suitable | Suitable | Suitable |
| | amount | amount | amount | amount |
| Purified water | Balance | Balance | Balance | Balance |
| Total | 100 | 100 | 100 | 100 |
| pH | 7.1 | 7.1 | 7.1 | 7.1 |
| Weight change per | 3.822 | 5.414 | 2.229 | 2.548 |
| unit volume (mg/cm³) | | | | |

For the heat treatment in Comparative Examples 23, 24 and Examples 88, 89, the glass vials were left to stand quietly at 50° C. for 7 days.

TABLE 34

| | Comparative | Comparative | | Comparative | |
|---|---|---|---|---|---|
| Unit: w/v % | Example 25 | Example 26 | Example 90 | Example 27 | Example 91 |
| (A) Gellan gum | — | 0.1 | 0.1 | — | — |
| (A) Xanthan gum | — | — | — | 0.14 | 0.14 |
| (B) Boric acid | — | — | 0.6 | — | 0.6 |
| (B) Borax | — | — | 0.02 | — | 0.02 |
| Sodium edetate | 0.1 | 0.1 | 0.1 | 0.1 | 0.1 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.0 | 7.0 | 7.0 | 7.0 | 7.0 |
| Weight change per | 6.688 | 7.325 | 2.229 | 7.006 | 5.096 |
| unit volume (mg/cm³) | | | | | |

For the heat treatment in Comparative Examples 25 to 27 and Examples 90, 91, the glass vials were left to stand quietly at 75° C. for 3 days.

TABLE 35

| | Comparative | Comparative | |
|---|---|---|---|
| Unit: w/v % | Example 28 | Example 29 | Example 92 |
| (A) Flavin adenine | — | 0.05 | 0.05 |
| dinucleotide sodium | | | |
| (B) Boric acid | — | — | 0.7 |
| (B) Borax | — | — | 0.01 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 5.2 | 5.2 | 5.0 |
| Weight change per | 3.503 | 4.459 | 2.548 |
| unit volume (mg/cm³) | | | |

For the heat treatment in Comparative Examples 28, 29 and Example 92, the glass vials were left to stand quietly at 50° C. for 12 days.

TABLE 36

| | Comparative | Comparative | |
|---|---|---|---|
| Unit: w/v % | Example 30 | Example 31 | Example 93 |
| (A) Panthenol | — | 0.1 | 0.1 |
| (B) Boric acid | — | — | 1.2 |
| (B) Borax | — | — | 0.4 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 5 | 5 | 5 |
| Weight change per | 4.777 | 5.732 | 3.503 |
| unit volume (mg/cm³) | | | |

For the heat treatment in Comparative Examples 30, 31 and Example 93, the glass vials were left to stand quietly at 50° C. for 17 days.

TABLE 37

| | Comparative | Comparative | |
|---|---|---|---|
| Unit: w/v % | Example 23 | Example 32 | Example 94 |
| (A) Castor oil | — | 0.02 | 0.02 |
| (A) Polyoxyethylene | — | 1.1 | 1.1 |
| hydrogenated | | | |
| castor oil 60 | | | |
| (B) Boric acid | — | — | 0.6 |
| (B) Borax | — | — | 0.02 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.1 | 7.1 | 7.1 |
| Weight change per | 3.822 | 9.236 | 0.955 |
| unit volume (mg/cm³) | | | |

For the heat treatment in Comparative Examples 23, 32 and Example 94, the glass vials were left to stand quietly at 50° C. for 7 days.

TABLE 38

| | Comparative | Comparative | |
|---|---|---|---|
| Unit: w/v % | Example 33 | Example 34 | Example 95 |
| (A) Refined lanolin | — | 0.05 | 0.05 |
| (A) Polyoxyethylene | — | 1 | 1 |
| hydrogenated | | | |
| castor oil 60 | | | |
| (B) Boric acid | — | — | 0.6 |
| (B) Borax | — | — | 0.02 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.1 | 7.0 | 7.0 |
| Weight change per | 4.140 | 5.096 | 2.866 |
| unit volume (mg/cm³) | | | |

For the heat treatment in Comparative Examples 33, 34 and Example 95, the glass vials were left to stand quietly at 50° C. for 12 days.

TABLE 39

| | Comparative | Comparative | Example | Example | Comparative | Example | Comparative | Example | Example |
|---|---|---|---|---|---|---|---|---|---|
| Unit: w/v % | Example 23 | Example 35 | 96 | 97 | Example 36 | 98 | Example 37 | 99 | 100 |
| (A) Polyoxyethylene | — | 0.4 | 0.4 | 0.4 | — | — | — | — | — |
| hydrogenated castor | | | | | | | | | |
| oil 60 | | | | | | | | | |
| (A) Polypxyl 40 stearate | — | — | — | — | 0.08 | 0.08 | — | — | — |
| (A) Poloxamer 407 | — | — | — | — | — | — | 0.5 | 0.5 | 0.5 |
| (B) Boric acid | — | — | 1.2 | — | — | 0.6 | — | 0.4 | 0.6 |
| (B) Borax | — | — | 0.2 | — | — | 0.02 | — | 0.015 | 0.02 |
| (B) Sodium hydrogen phosphate | — | — | — | 0.8 | — | — | — | — | — |
| (B) Sodium dihydrogen | — | — | — | 0.1 | — | — | — | — | — |
| phosphate | | | | | | | | | |
| (B) Sodium citrate | — | — | — | 0.5 | — | — | — | — | — |
| (B) 2-amino-2-hydroxymethyl- | — | — | — | — | — | — | — | — | 0.5 |
| 1,3-propanediol | | | | | | | | | |
| (B) Sodium hydrogen carbonate | — | — | — | — | — | — | — | — | 0.2 |
| Hydrochloric acid | Suitable | Suitable | Suitable | Suitable | Suitable | Suitable | Suitable | Suitable | Suitable |
| | amount | amount | amount | amount | amount | amount | amount | amount | amount |
| Sodium hydroxide | Suitable | Suitable | Suitable | Suitable | Suitable | Suitable | Suitable | Suitable | Suitable |
| | amount | amount | amount | amount | amount | amount | amount | amount | amount |
| Purified water | Balance | Balance | Balance | Balance | Balance | Balance | Balance | Balance | Balance |
| Total | 100 | 100 | 100 | 100 | 100 | 100 | 100 | 100 | 100 |
| pH | 7.1 | 7.1 | 7.1 | 7.0 | 7.0 | 7.0 | 7.0 | 7.0 | 7.0 |
| Weight change per unit volume | 3.822 | 7.643 | 1.911 | 1.274 | 5.096 | 1.274 | 6.688 | 2.229 | 2.229 |
| (mg/cm³) | | | | | | | | | |

For the heat treatment in Comparative Examples 23, 35 to 37 and Examples 96 to 100, the glass vials were left to stand quietly at 50° C. for 7 days.

TABLE 40

| | Comparative | Comparative | |
|---|---|---|---|
| Unit: w/v % | Example 23 | Example 38 | Example 101 |
| (A) Diphenhydramine | — | 0.05 | 0.05 |
| hydrochloride | | | |
| (B) Boric acid | — | — | 0.6 |
| (B) Borax | — | — | 0.02 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.1 | 7.0 | 7.0 |
| Weight change per unit | 3.822 | 5.096 | 2.548 |
| volume (mg/cm³) | | | |

For the heat treatment in Comparative Examples 23, 38 and Example 101, the glass vials were left to stand quietly at 50° C. for 7 days.

TABLE 41

| | Comparative | Comparative | |
|---|---|---|---|
| Unit: w/v % | Example 39 | Example 40 | Example 102 |
| (1) Tranilast | — | 0.5 | 0.5 |
| (A) Polyvinylpyrrolidone k25 | — | 2.5 | 2.5 |
| (B) Boric acid | — | — | 0.5 |
| (B) Borax | — | — | 0.2 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.9 | 7.9 | 7.9 |
| Weight change per unit | 5.096 | 6.051 | 3.503 |
| volume (mg/cm³) | | | |

For the heat treatment in Comparative Examples 39, 40 and Example 102, the glass vials were left to stand quietly at 50° C. for 7 days.

TABLE 42

| | Comparative | Comparative | Example | Comparative | Example |
|---|---|---|---|---|---|
| Unit: w/v % | Example 41 | Example 42 | 103 | Example 43 | 104 |
| (A) Tranilast | — | 0.5 | 0.5 | 0.5 | 0.5 |
| (A) Polyvinylpyrrolidone k90 | — | — | — | 2.5 | 2.5 |
| (B) Boric acid | — | — | 0.6 | — | 1.1 |
| (B) Borax | — | — | 0.04 | — | 0.6 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.9 | 8.0 | 8.0 | 7.9 | 7.9 |
| Weight change per unit volume | 6.369 | 8.280 | 5.732 | 7.325 | 2.866 |
| (mg/cm³) | | | | | |

For the heat treatment in Comparative Examples 41 to 43 and Examples 103, 104, the glass vials were left to stand quietly at 50° C. for 12 days.

TABLE 43

| | Comparative | Comparative | Example |
|---|---|---|---|
| Unit: w/v % | Example 44 | Example 45 | 105 |
| (A) Ketotifen fumarate | — | 0.069 | 0.069 |
| (B) Boric acid | — | — | 0.7 |
| (B) Borax | — | — | 0.01 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 5.3 | 5.3 | 5.3 |
| Weight change per unit volume | 1.274 | 3.185 | 0.955 |
| (mg/cm³) | | | |

For the heat treatment in Comparative Examples 44, 45 and Example 105, the glass vials were left to stand quietly at 75° C. for 3 days.

TABLE 44

| | Comparative | Comparative | Example | Comparative | Example |
|---|---|---|---|---|---|
| Unit: w/v % | Example 33 | Example 46 | 106 | Example 47 | 107 |
| (A) Chlorhexidine gluconate | — | 0.005 | 0.005 | — | — |
| (A) Potassium sorbate | — | — | — | 0.1 | 0.1 |
| (B) Boric acid | — | — | 0.6 | — | 0.6 |
| (B) Borax | — | — | 0.02 | — | 0.02 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.1 | 7.0 | 7.0 | 7.0 | 7.0 |
| Weight change per unit volume | 4.140 | 4.777 | 3.822 | 6.051 | 3.822 |
| (mg/cm³) | | | | | |

For the heat treatment in Comparative Examples 33, 46, 47 and Examples 106, 107, the glass vials were left to stand quietly at 50° C. for 12 days.

TABLE 45

| | Comparative | Comparative | | Comparative | | Comparative | |
|---|---|---|---|---|---|---|---|
| | Example | Example | Example | Example | Example | Example | Example |
| Unit: w/v % | 23 | 48 | 108 | 49 | 109 | 50 | 110 |
| (A) Methyl cellulose | — | 0.4 | 0.4 | — | — | — | — |
| (A) Polyvinylpyrrolidone k90 | — | — | — | 3.0 | 3.0 | — | — |
| (A) Carboxyvinyl polymer | — | — | — | — | — | 0.3 | 0.3 |
| (B) Boric acid | — | — | 0.6 | — | 0.6 | — | 0.6 |
| (B) Borax | — | — | 0.02 | — | 0.02 | — | 0.02 |
| Hydrochloric acid | Suitable | Suitable | Suitable | Suitable | Suitable | Suitable | Suitable |
| | amount | amount | amount | amount | amount | amount | amount |
| Sodium hydroxide | Suitable | Suitable | Suitable | Suitable | Suitable | Suitable | Suitable |
| | amount | amount | amount | amount | amount | amount | amount |
| Purified water | Balance | Balance | Balance | Balance | Balance | Balance | Balance |
| Total | 100 | 100 | 100 | 100 | 100 | 100 | 100 |
| pH | 7.1 | 7.1 | 7.0 | 7.1 | 7.0 | 7.0 | 7.0 |
| Weight change per unit volume | 3.822 | 6.051 | 1.911 | 6.688 | 1.911 | 8.599 | 2.229 |
| (mg/cm³) | | | | | | | |

For the heat treatment in Comparative Examples 23, 48 to 50 and Examples 108 to 110, the glass vials were left to stand quietly at 50° C. for 7 days.

TABLE 46

| | Comparative | Comparative | Example | Comparative | Example |
|---|---|---|---|---|---|
| Unit: w/v % | Example 33 | Example 51 | 111 | Example 52 | 112 |
| (A) Carboxymethyl cellulose | — | 0.4 | 0.4 | — | — |
| sodium | | | | | |
| (A) Polyvinylpyrrolidone k25 | — | — | — | 2.5 | 2.5 |
| (B) Boric acid | — | — | 0.6 | — | 1.0 |
| (B) Borax | — | — | 0.02 | — | 0.2 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.1 | 7.0 | 7.1 | 7.0 | 7.1 |
| Weight change per unit volume | 4.140 | 5.414 | 2.866 | 5.096 | 3.135 |
| (mg/cm³) | | | | | |

For the heat treatment in Comparative Examples 33, 51, 52 and Examples 111, 112, the glass vials were left to stand quietly at 50° C. for 12 days.

TABLE 47

| | Comparative | Comparative | Example | Comparative | Example |
|---|---|---|---|---|---|
| Unit: w/v % | Example 23 | Example 53 | 113 | Example 54 | 114 |
| (A) Propylene glycol | — | 1.0 | 1.0 | — | — |
| (A) D-Mannitol | — | — | — | 1.0 | 1.0 |
| (B) Boric acid | — | — | 0.6 | — | 0.6 |
| (B) Borax | — | — | 0.02 | — | 0.02 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.1 | 7.1 | 7.0 | 7.0 | 7.0 |
| Weight change per unit volume | 3.822 | 4.777 | 2.229 | 5.414 | 0.318 |
| (mg/cm³) | | | | | |

For the heat treatment in Comparative Examples 23, 53, 54 and Examples 113, 114, the glass vials were left to stand quietly at 50° C. for 7 days.

TABLE 48

| | Comparative | Comparative | Example |
|---|---|---|---|
| Unit: w/v % | Example 33 | Example 55 | 115 |
| (A) Glycerin | — | 2.0 | 2.0 |
| (B) Boric acid | — | — | 0.6 |
| (B) Borax | — | — | 0.02 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.1 | 7.0 | 7.0 |
| Weight change per unit volume | 4.140 | 5.414 | 3.503 |
| (mg/cm³) | | | |

For the heat treatment in Comparative Examples 33, 55 and Example 115, the glass vials were left to stand quietly at 50° C. for 12 days.

TABLE 49

| | Comparative | Comparative | Example |
|---|---|---|---|
| Unit: w/v % | Example 56 | Example 57 | 116 |
| (A) Allantoin | — | 0.3 | 0.3 |
| (B) Boric acid | — | — | 0.7 |
| (B) Borax | — | — | 0.01 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 5 | 5 | 5 |
| Weight change per unit volume | 2.866 | 3.503 | 1.592 |
| (mg/cm³) | | | |

For the heat treatment in Comparative Examples 56, 57 and Example 116, the glass vials were left to stand quietly at 50° C. for 7 days.

TABLE 50

| | Comparative | Comparative | Example |
|---|---|---|---|
| Unit: w/v % | Example 39 | Example 58 | 117 |
| (A) Sodium azulenesulfonate | — | 0.02 | 0.02 |
| (B) Boric acid | — | — | 0.6 |
| (B) Borax | — | — | 0.04 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.9 | 8.1 | 8.0 |
| Weight change per unit volume | 5.096 | 5.414 | 0.000 |
| (mg/cm³) | | | |

For the heat treatment in Comparative Examples 39, 58 and Example 117, the glass vials were left to stand quietly at 50° C. for 7 days.

TABLE 51

| | Comparative | Comparative | Example | Comparative | Example |
|---|---|---|---|---|---|
| Unit: w/v % | Example 30 | Example 59 | 118 | Example 60 | 119 |
| (A) Berberine chloride | — | 0.025 | 0.025 | — | — |
| (A) Zinc sulfate | — | — | — | 0.25 | 0.25 |
| (B) Boric acid | — | — | 0.7 | — | 0.7 |
| (B) Borax | — | — | 0.01 | — | 0.01 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 5 | 5 | 5 | 5 | 5 |
| Weight change per unit volume | 4.777 | 5.414 | 4.140 | 5.096 | 4.140 |
| (mg/cm³) | | | | | |

For the heat treatment in Comparative Examples 30, 59, 60 and Examples 118, 119, the glass vials were left to stand quietly at 50° C. for 17 days.

TABLE 52

| | Comparative | Comoarative | Example |
|---|---|---|---|
| Unit: w/v % | Example 39 | Example 61 | 120 |
| (A) Sulfamethoxazole sodium | — | 4.0 | 4.0 |
| (B) Boric acid | — | — | 0.6 |
| (B) Borax | — | — | 0.04 |
| Hydrochloric acid | Suitable | Suitable | Suitable |
| | amount | amount | amount |
| Sodium hydroxide | Suitable | Suitable | Suitable |
| | amount | amount | amount |
| Purified water | Balance | Balance | Balance |
| Total | 100 | 100 | 100 |
| pH | 7.9 | 8.0 | 8.0 |
| Weight change per unit volume | 5.096 | 5.732 | 4.140 |
| (mg/cm³) | | | |

For the heat treatment in Comparative Examples 39, 61 and Example 120, the glass vials were left to stand quietly at 50° C. for 7 days.

TABLE 53

| | Comparative | Comparative | Example | Comparative | Example |
|---|---|---|---|---|---|
| Unit: w/v % | Example 23 | Example 62 | 121 | Example 63 | 122 |
| (A) Eucalyptus oil | — | 0.005 | 0.005 | — | — |
| (A) Bergamot oil | — | — | — | 0.002 | 0.002 |
| (A) Polyoxyethylene | — | 0.1 | 0.1 | 0.1 | 0.1 |
| hydrogenated castor oil 60 | | | | | |
| (B) Boric acid | — | — | 0.6 | — | 0.6 |
| (B) Borax | — | — | 0.02 | — | 0.02 |
| Hydrochloric acid | Suitable | Suitable | Suitable | Suitable | Suitable |
| | amount | amount | amount | amount | amount |
| Sodium hydroxide | Suitable | Suitable | Suitable | Suitable | Suitable |
| | amount | amount | amount | amount | amount |
| Purified water | Balance | Balance | Balance | Balance | Balance |
| Total | 100 | 100 | 100 | 100 | 100 |
| pH | 7.1 | 7.0 | 7.0 | 7.0 | 7.0 |
| Weight change per unit volume | 3.822 | 4.140 | 1.592 | 4.777 | 0.318 |
| (mg/cm³) | | | | | |

For the heat treatment in Comparative Examples 23, 62, 63 and Examples 121, 122, the glass vials were left to stand quietly at 50° C. for 7 days.

<Examples of Preparation of Aqueous Ophthalmic Compositions Held in Containers>

Aqueous ophthalmic compositions of the following Tables 54 to 57 (Tables 56 and 57 each extend in two pages) are prepared. The container main body part made of a PET-containing resin was filled with each of formulation examples 2, 3, 6, 9, 10, 15, 18 to 21; a perforated inside plug made of a PBT-containing resin was attached to an opening of the main body part; and a lid made of a PP-containing resin was put thereon. The container main body part made of a PP-containing resin was filled with each of formulation examples 7 to 8; a perforated inside plug made of a PBT-containing resin was attached to an opening of the main body part; and a lid made of an ABS-containing resin was put thereon. The container main body part made of an ethylene vinyl acetate copolymer-containing resin was filled with each of formulation examples 4, 14; a perforated inside plug made of a PBT-containing resin was attached to an opening of the main body part; and a lid made of a PE-containing resin was put thereon. The container main body part made of a PBT-containing resin was filled with each of formulation examples 1, 5, 17; a perforated inside plug made of a PE-containing resin was attached to an opening of the main body part; and a lid made of a PS-containing resin was put thereon. The container main body part made of a PE-containing resin was filled with each of formulation examples 11, 13, 16; a perforated inside plug made of a PBT-containing resin was attached to an opening of the main body part; and a lid made of a PP-containing resin was put thereon. A container in which the main body part for storing the aqueous ophthalmic composition and the opening are made of the same PBT-containing resin was filled with formulation example 12, and a lid made of a PP-containing resin was put thereon. The unit of numerical values in the formulation examples is “w/v %”.

TABLE 54

| | Formulation 1 | Formulation 2 | Formulation 3 | Formulation 4 | Formulation 5 | Formulation 6 |
|---|---|---|---|---|---|---|
| | Eye drops | Eye drops | Eye drops | Eye drops | Eye drops | Eye drops |
| Tetrahydrozoline | | 0.05 | | 0.05 | | 0.03 |
| hydrochloride | | | | | | |
| Chlorpheniramine maleate | 0.01 | 0.03 | | 0.03 | | 0.03 |
| Dipotassium glycyrrhizinate | | | | | | 0.25 |
| Allantoin | | 0.2 | | | | |
| Zinc sulfate | | 0.05 | | | | |
| Neostigmine methylsulfate | 0.005 | 0.005 | | 0.005 | | |
| Pyridoxine hydrochloride | | 0.1 | | 0.1 | | |
| Tocopherol acetate | 0.015 | | | | | |
| Panthenol | | | | | | 0.1 |
| Sodium chondroitin sulfate | 0.05 | 0.05 | 0.5 | 0.5 | 0.05 | |
| Potassium L-aspartate | | 1 | 1 | 1 | | |
| Aminoethylsulfonic acid | 0.1 | | | 1 | | 1 |
| 1-menthol | 0.001 | 0.06 | 0.02 | 0.05 | 0.01 | 0.01 |
| d-camphor | | 0.01 | | 0.03 | 0.01 | 0.001 |
| dl-camphor | | | | 0.02 | | |
| d-borneol | 0.001 | | 0.01 | 0.02 | | 0.005 |
| Geraniol | 0.001 | | | | | |
| Sodium hyaluronate | | | 0.005 | | | 0.05 |
| Hypromellose | | 0.5 | 0.1 | 0.01 | 0.5 | |
| Hydroxyethyl cellulose | | 0.4 | | 0.01 | | |
| Glucose | 0.005 | 0.02 | | | | |
| Polyhexanide hydrochloride | | | | | 0.001 | |
| Benzalkonium chloride | 0.02 | | | 0.01 | | 0.005 |
| Phenylethyl alcohol | 0.05 | | | | | |
| Chlorobutanol | 0.05 | 0.2 | 0.2 | 0.1 | | |
| Sodium edetate | 0.2 | 0.1 | 0.1 | | | 0.05 |
| Sodium chloride | | | 0.1 | | 0.6 | |
| Potassium chloride | | | 0.2 | | 0.1 | |
| ε-aminocaproic acid | 0.3 | | | 1 | | 1 |
| Boric acid | | 0.8 | 0.8 | 0.2 | 1 | |
| Borax | | 0.05 | 0.1 | | 0.2 | |
| Sodium citrate | | | .0.5 | | | |
| Sodium hydrogen phosphate | | | | | 0.5 | |
| Polyoxyethylene hydrogenated | | 0.4 | 0.1 | | | 0.01 |
| castor oil | | | | | | |
| Polysorbate 80 | 0.1 | | 0.3 | | | |
| Propylene glycol | | 0.01 | | | | |
| Dextran | | | | 0.2 | | |
| Polyethylene glycol 40000 | | | | | 0.5 | |
| pH | 6 | 5 | 6.5 | 5.7 | 7.8 | 6 |

TABLE 55

| | | | | For- |
|---|---|---|---|---|
| | | | | mula- |
| | Formula- | Formula- | Formula- | tion 10 |
| | tion 7 | tion 8 | tion 9 | Eye |
| | Eye drops | Eye drops | Eye drops | wash |
| Chlorpheniramine maleate | | | | 0.003 |
| Dipotassium glycyrrhizinate | | | | 0.025 |
| Zinc sulfate | | | | 0.01 |
| Pyridoxine hydrochloride | | | | 0.01 |
| Cyanocobalamine | | | | 0.001 |
| Tocopherol acetate | | | | 0.005 |
| Sodium chondroitin sulfate | | 0.5 | 0.05 | 0.05 |
| Potassium L-aspartate | | | 0.5 | 0.1 |
| Aminoethylsulfonic acid | | | | 0.02 |
| 1-menthol | 0.005 | 0.02 | 0.05 | |
| d-camphor | | 0.01 | | |
| dl-camphor | | | | 0.08 |
| d-borneol | | | 0.005 | 0.002 |
| Geraniol | 0.005 | | | 0.008 |
| Sodium hyaluronate | 0.1 | 0.01 | | 0.001 |
| Hypromellose | 0.005 | 0.1 | 0.01 | |
| Hydroxyethyl cellulose | 0.005 | | | |
| Glucose | 0.001 | | 0.1 | |
| Potassium sorbate | | | 0.1 | |
| Polyhexanide hydrochloride | | 0.0001 | | |
| Chlorhexidine gluconate | 0.005 | | | |
| Chlorobutanol | | | 0.3 | |
| Sodium edetate | 0.01 | 0.001 | 0.002 | 0.02 |
| Sodium chloride | 1 | 0.5 | 0.01 | |
| Potassium chloride | 0.01 | 0.3 | 0.8 | |
| Calcium chloride | | 0.005 | | |
| Sodium hydrogen carbonate | | 0.05 | | |
| Boric acid | 0.05 | 0.5 | 1.5 | 2 |
| Borax | | 0.01 | 0.1 | 0.08 |
| 2-amino-2-hydroxymethyl- | | | 0.1 | |
| 1,3-propanediol | | | | |
| Polyoxyethylene | 0.5 | 0.01 | 0.3 | 0.1 |
| hydrogenated castor | | | | |
| oil | | | | |
| Poloxamer | | 0.5 | | |
| Polysorbate 80 | | 0.01 | | 0.6 |
| pH | 7 | 7 | 7 | 5.5 |

TABLE 56

| | Formulation 11 | Formulation 12 | Formulation 13 | Formulation 14 | Formulation 15 | Formulation 16 |
|---|---|---|---|---|---|---|
| | Eye drops | Eye drops | Eye drops | Eye drops | Eye drops | Eye drops |
| Boric acid | 1.2 | — | — | 0.8 | 0.2 | — |
| Borax | 0.3 | — | — | — | 0.4 | — |
| Sodium citrate | 0.02 | — | — | 0.8 | 0.5 | — |
| Anhydrous citric acid | 0.01 | — | — | — | — | — |
| Sodium hydrogen phosphate | — | 0.8 | 1 | — | — | — |
| Sodium dihydrogen phosphate | — | — | 0.2 | — | — | — |
| dihydrate | | | | | | |
| 2-amino-2-hydroxymethyl- | 0.1 | — | — | 1 | — | — |
| 1,3-propanediol | | | | | | |
| Tetrahydrozoline hydrochloride | — | — | — | — | — | 0.01 |
| Naphazoline hydrochloride | — | — | — | 0.003 | — | — |
| Chlorpheniramine maleate | 0.03 | — | — | — | — | 0.03 |
| Dipotassium glycyrrhizinate | 0.125 | 0.05 | — | — | — | 0.25 |
| Sodium azulenesulfonate | — | — | — | — | — | — |
| Berberine sulfate | — | — | — | — | — | — |
| Zinc sulfate | — | — | — | — | — | — |
| Lysozyme chloride | — | — | — | — | — | — |
| Sodium cromoglicate | — | — | — | — | — | — |
| Neostigmine methylsulfate | — | — | — | — | — | 0.05 |
| Pyridoxine hydrochloride | 0.01 | — | — | — | — | 0.1 |
| Cyanocobalamine | — | — | — | — | — | — |
| Tocopherol acetate | — | 0.04 | — | 0.05 | — | — |
| Panthenol | — | — | — | — | — | — |
| Flavin adenine dinucleotide sodium | — | — | — | — | — | 0.05 |
| Retinol palmitate | — | 10,000 units | — | 100,000 units | — | — |
| Sodium chondroitin sulfate | 0.5 | — | — | 0.05 | 0.25 | 0.5 |
| Potassium L-aspartate | 0.2 | — | — | — | 0.2 | 0.1 |
| Aminoethylsulfonic acid | 0.1 | — | — | — | 0.6 | 1 |
| 1-menthol | 0.01 | — | — | — | 0.02 | — |
| d-camphor | 0.005 | — | — | — | — | — |
| dl-camphor | — | — | — | — | — | — |
| d-borneol | 0.001 | — | — | — | — | — |
| Geraniol | — | — | — | — | — | — |
| Cool mint oil | — | — | 0.015 | — | — | — |
| Peppermint oil | — | — | 0.015 | — | — | 0.01 |
| Eucalyptus oil | — | — | — | — | — | 0.005 |
| Bergamot oil | — | — | 0.02 | — | — | — |
| Sodium hyaluronate | 0.003 | 0.001 | — | — | — | — |
| Hypromellose | 0.3 | 0.01 | — | — | 0.05 | — |
| Hydroxyethyl cellulose | — | — | — | — | 0.4 | 0.05 |
| Polyvinyl alcohol | — | — | 1 | — | — | — |
| Polyvinylpyrrolidone 25 | — | 0.1 | — | — | — | — |
| Polyvinylpyrrolidone 30 | 0.25 | — | — | — | — | — |
| Polyvinylpyrrolidone 90 | — | — | — | 0.05 | — | — |
| Pranoprofen | — | — | — | — | — | — |
| Potassium sorbate | — | — | — | 0.1 | 0.05 | — |
| Polyhexanide hydrochloride | — | — | — | — | 0.00008 | — |
| Benzalkonium chloride | — | — | — | — | 0.005 | 0.001 |
| Chlorhexidine gluconate | — | — | — | — | 0.002 | — |
| Zinc chloride | — | — | 0.001 | — | — | — |
| Chlorobutanol | — | — | — | — | 0.4 | 0.01 |
| Sodium edetate | 0.1 | — | — | 0.01 | 0.05 | 0.005 |
| Sodium chloride | 0.2 | — | 0.4 | 0.3 | 0.1 | — |
| Potassium chloride | — | — | 0.1 | — | 0.1 | — |
| Calcium chloride | — | — | 0.01 | — | — | — |
| Magnesium sulfate | — | — | 0.01 | — | — | — |
| Sodium hydrogen carbonate | — | — | 0.02 | — | — | — |
| Epsilon-aminocaproic acid | — | — | — | — | — | 0.1 |
| Polyoxyethylene hydrogenated | — | 0.07 | — | — | 0.4 | 1 |
| castor oil 60 | | | | | | |
| Polyoxyethylene hydrogenated | — | — | — | 0.15 | — | — |
| castor oil 40 | | | | | | |
| Polyoxyethylene castor oil 3 | — | — | — | — | — | — |
| Polyoxyethylene castor oil 10 | — | 0.1 | — | — | — | — |
| Polyoxyethylene castor oil 35 | — | — | — | 0.01 | — | — |
| Poloxamer 407 | — | — | 0.08 | 0.1 | 0.01 | — |
| POE (200) POP (70) glycol | — | 0.35 | — | 3 | — | — |
| Poloxamer 188 | 0.1 | — | — | — | — | — |
| Poloxamer 235 | — | — | — | — | — | — |
| Polyoxyl 40 stearate | — | — | — | — | 0.08 | — |
| Polyoxyl 140 stearate | — | — | — | — | — | — |
| Sesame oil | — | — | — | 0.1 | — | — |
| Castor oil | — | 0.05 | — | 0.1 | — | — |
| Polysorbate 80 | 0.1 | 0.01 | — | — | 0.2 | 1 |
| Propylene glycol | 0.3 | — | 0.1 | 1 | 0.8 | — |
| Glycerin | — | 1.2 | — | — | 0.3 | 2 |
| Dibutylhydroxytoluene | — | — | — | — | — | — |
| Dextran70 | — | — | — | — | — | 0.01 |
| Polyethylene glycol 4000 | — | — | 0.1 | — | 1 | — |
| Ethanol | — | — | — | — | — | 0.1 |
| Xylitol | — | — | — | — | — | — |
| Hydrochloric acid | Suitable amount | Suitable amount | Suitable amount | Suitable amount | Suitable amount | Suitable amount |
| Sodium hydroxide | Suitable amount | Suitable amount | Suitable amount | Suitable amount | Suitable amount | Suitable amount |
| Purified water | Balance | Balance | Balance | Balance | Balance | Balance |
| pH | 7 | 5.2 | 7.8 | 7.1 | 7 | 5 |

TABLE 57

| | Formulation 17 | Formulation 18 | Formulation 19 | Formulation 20 | Formulation 21 |
|---|---|---|---|---|---|
| | Eye drops | Eye drops | Eye drops | Eye drops | Eye drops |
| Boric acid | 0.5 | 0.5 | 0.3 | 2 | 1 |
| Borax | — | — | 0.01 | 0.4 | |
| Sodium citrate | — | — | — | — | — |
| Anhydrous citric acid | — | — | — | — | — |
| Sodium hydrogen phosphate | 0.5 | — | — | — | — |
| Sodium dihydrogen phosphate dihydrate | — | — | — | — | — |
| 2 amino-2-hydroxymethyl-1,3-propanediol | 3 | — | 1 | — | 0.1 |
| Tetrahydrozoline hydrochloride | 0.05 | 0.05 | 0.03 | — | 0.01 |
| Naphazoline hydrochloride | — | — | — | — | — |
| Chlorpheniramine maleate | 0.03 | 0.03 | 0.015 | 0.03 | 0.03 |
| Dipotassium glycyrrhizinate | 0.25 | 0.1 | — | — | — |
| Sodium azulenesulfonate | 0.02 | — | — | — | — |
| Berberine sulfate | 0.01 | — | — | — | — |
| Zinc sulfate | — | 0.05 | — | — | — |
| Lysozyme chloride | 0.5 titer | — | — | — | — |
| Sodium cromoglicate | — | — | — | 1 | — |
| Neostigmine methylsulfate | — | 0.005 | 0.001 | — | 0.005 |
| Pyridoxine hydrochloride | 0.08 | 0.1 | 0.05 | — | 0.03 |
| Cyanocobalamine | — | 0.02 | — | — | — |
| Tocopherol acetate | — | | 0.05 | — | 0.05 |
| Panthenol | — | 0.05 | — | — | — |
| Flavin adenine dinucleotide sodium | — | — | — | — | — |
| Retinol palmitate | — | — | 40,000 units | — | 50,000 units |
| Sodium chondroitin sulfate | — | 0.5 | 0.05 | 0.5 | — |
| Potassium L-aspartate | — | 1 | 0.8 | — | 0.5 |
| Aminoethylsulfonic acid | — | 1 | 0.1 | — | — |
| 1-menthol | — | 0.01 | 0.04 | 0.015 | — |
| d-camphor | — | — | 0.01 | — | — |
| d1-camphor | — | — | 0.01 | 0.006 | — |
| d-borneol | 0.015 | 0.02 | 0.001 | 0.008 | — |
| Geraniol | — | 0.001 | — | — | — |
| Cool mint oil | — | — | — | — | — |
| Peppermint oil | — | — | — | — | — |
| Eucalyptus oil | — | — | 0.0005 | — | — |
| Bergamot oil | — | 0.002 | — | — | — |
| Sodium hyaluronate | — | 0.02 | — | — | — |
| Hypromellose | 0.2 | | | | |
| Hydroxyethyl cellulose | — | 0.1 | — | — | 0.1 |
| Polyvinyl alcohol | — | — | — | — | — |
| Polyvinylpyrrolidone 25 | — | — | — | — | — |
| Polyvinylpyrrolidone 30 | — | — | — | — | — |
| Polyvinylpyrrolidone 90 | — | 0.05 | — | — | — |
| Pranoprofen | — | — | — | 0.05 | — |
| Potassium sorbate | — | — | — | 0.1 | — |
| Polyhexanide hydrochloride | — | — | 0.00005 | 0.00004 | — |
| Benzalkonium chloride | — | 0.01 | 0.01 | 0.01 | 0.003 |
| Chlorhexidine gluconate | — | — | — | 0.002 | — |
| Zinc chloride | — | — | — | — | — |
| Chlorobutanol | — | 0.5 | 0.05 | — | — |
| Sodium edetate | 0.13 | 0.01 | 0.08 | 0.06 | 0.1 |
| Sodium chloride | — | — | 0.3 | — | 0.2 |
| Potassium chloride | — | — | — | — | — |
| Calcium chloride | — | — | — | — | — |
| Magnesium sulfate | — | — | — | — | — |
| Sodium hydrogen carbonate | — | — | — | — | — |
| Epsilon-aminocaproic acid | — | 0.8 | 2 | — | — |
| Polyoxyethylene hydrogeneted | 0.15 | — | 0.5 | 0.05 | 0.05 |
| castor oil 60 | | | | | |
| Polyoxyethylene hydrogenated | — | — | — | 0.1 | — |
| castor oil 40 | | | | | |
| Polyoxyethylene castor oil 3 | — | — | 0.1 | — | — |
| Polyoxyethylene castor oil 10 | 0.8 | — | — | — | — |
| polyoxyethylene castor oil 35 | — | — | — | — | — |
| Poloxamer 407 | — | — | — | — | — |
| POE (200) POP (70) glycol | — | — | 1 | — | 5 |
| Poloxamer 188 | | — | — | — | — |
| Poloxamer 235 | 0.05 | — | — | — | — |
| Polyoxyl 40 stearate | 0.1 | — | — | — | 0.05 |
| Polyoxyl 140 stearate | — | 0.1 | — | — | — |
| Sesame oil | — | — | 0.01 | — | — |
| Castor oil | — | — | 0.01 | — | — |
| Polysorbate 80 | — | 0.5 | 0.5 | 0.3 | 0.05 |
| Propylene glycol | 2 | 0.1 | 1 | — | 0.5 |
| Glycerin | — | — | 0.6 | — | — |
| Dibutylhydroxytoluene | 0.001 | — | 0.005 | 0.005 | 0.005 |
| Dextran 70 | — | 0.05 | — | — | — |
| Polyethylene glycol 4000 | | | | | |
| Ethanol | — | — | — | — | — |
| Xylitol | — | — | — | — | 0.05 |
| Hydrochloric acid | Suitable amount | Suitable amount | Suitable amount | Suitable amount | Suitable amount |
| Sodium hydroxide | Suitable amount | Suitable amount | Suitable amount | Suitable amount | Suitable amount |
| Purified water | Balance | Balance | Balance | Balance | Balance |
| pH | 6.5 | 5 | 7 | 7 | 7 |

POE (200) POP (70) represents polyoxyethylene (200) polyoxypropylene (70) glycol.

1. An aqueous ophthalmic composition, comprising
(A) at least one surfactant selected from the group consisting of polyoxyethylene castor oil, polyoxyethylene hydrogenated castor oil, polyoxyethylene polyoxypropylene glycol, polyoxyl stearate, and polysorbate 80;
at least one cooling and/or refreshing agent of 0.015-0.1 w/v % selected from the group consisting of eucalyptus oil and bergamot oil, and menthol; and
(B) a buffering agent;
wherein (C) the aqueous ophthalmic composition is held in a container in which a part of a surface or the whole surface that is brought into contact with the aqueous ophthalmic composition is molded with a resin containing polybutylene terephthalate.

2. The aqueous ophthalmic composition according to claim 1, further comprising at least one polyhydric alcohol selected from the group consisting of propylene glycol, glycerin, polyethylene glycol, and mannitol.

3. The aqueous ophthalmic composition according to claim 1, further comprising chlorobutanol.

4-6. (canceled)

7. The aqueous ophthalmic composition according to claim 1, further comprising at least one thickening component selected from the group consisting of carboxymethyl cellulose, methyl cellulose, hydroxyethyl cellulose, vinyl-based polymer compound, and salts thereof.

8. The aqueous ophthalmic composition according to claim 1, further comprising at least one decongestants selected from the group consisting of tetrahydrozoline hydrochloride, naphazoline hydrochloride, naphazoline nitrate, epinephrine, epinephrine hydrochloride, ephedrine hydrochloride, phenylephrine hydrochloride, and methylephedrine hydrochloride.

9. A method for imparting a function of inhibiting change in weight of a polybutylene terephthalate-containing resin container to an aqueous composition by allowing
(A) at least one surfactant selected from the group consisting of polyoxyethylene castor oil, polyoxyethylene hydrogenated castor oil, polyoxyethylene polyoxypropylene glycol, polyoxyl stearate, and polysorbate 80;
at least one cooling and/or refreshing agent of 0.015-0.1 w/v % selected from the group consisting of eucalyptus oil, bergamot oil, and menthol; and
(B) a buffering agent;
to coexist in the aqueous composition.

10. The method according to claim 9, wherein said aqueous composition further comprising at least one polyhydric alcohol selected from the group consisting of propylene glycol, glycerin, polyethylene glycol, and mannitol.

11. The method according to claim 9, wherein said aqueous composition further comprising chlorobutanol.

12. The method according to claim 9, wherein said aqueous composition further comprising at least one thickening component selected from the group consisting of carboxymethyl cellulose, methyl cellulose, hydroxyethyl cellulose, vinyl-based polymer compound, and salts thereof.

13. The method according to claim 9, wherein said aqueous composition further comprising at least one decongestants selected from the group consisting of tetrahydrozoline hydrochloride, naphazoline hydrochloride, naphazoline nitrate, epinephrine, epinephrine hydrochloride, ephedrine hydrochloride, phenylephrine hydrochloride, and methylephedrine hydrochloride.

14. A method for imparting a function of inhibiting wetting of a polybutylene terephthalate-containing resin container to an aqueous composition by allowing
(A) at least one surfactant selected from the group consisting of polyoxyethylene castor oil, polyoxyethylene hydrogenated castor oil, polyoxyethylene polyoxypropylene glycol, polyoxyl stearate, and polysorbate 80;
at least one cooling and/or refreshing agent of 0.015-0.1 w/v % selected from the group consisting of eucalyptus oil, bergamot oil, and menthol; and
(B) a buffering agent;
to coexist in the aqueous composition.

15. The method according to claim 14, wherein said aqueous composition further comprising at least one polyhydric alcohol selected from the group consisting of propylene glycol, glycerin, polyethylene glycol, and mannitol.

16. The method according to claim 14, wherein said aqueous composition further comprising chlorobutanol.

17. The method according to claim 14, wherein said aqueous composition further comprising at least one thickening component selected from the group consisting of carboxymethyl cellulose, methyl cellulose, hydroxyethyl cellulose, vinyl-based polymer compound, and salts thereof.

18. The method according to claim 14, wherein said aqueous composition further comprising at least one decongestants selected from the group consisting of tetrahydrozoline hydrochloride, naphazoline hydrochloride, naphazoline nitrate, epinephrine, epinephrine hydrochloride, ephedrine hydrochloride, phenylephrine hydrochloride, and methylephedrine hydrochloride.
